(19) United States
(12) Patent Application Publication
(10) Pub. No.: US 2021/0338189 A1
(43) Pub. Date: Nov. 4, 2021

(54) AUSCULTATION SYSTEM

(71) Applicants: Marina VERNALIS, Silver Spring, MD (US); Brian J. BOOTH, Munster (CA); Simon MARTIN, Gatineau (CA); Jun ZHOU, Kanata (CA); Md Shahidul ISLAM, Ottawa (CA); Steven P. MORTON, Kanata (CA)

(72) Inventors: Marina VERNALIS, Silver Spring, MD (US); Brian J. BOOTH, Munster (CA); Simon MARTIN, Gatineau (CA); Jun ZHOU, Kanata (CA); Md Shahidul ISLAM, Ottawa (CA); Steven P. MORTON, Kanata (CA)

(21) Appl. No.: 17/306,888

(22) Filed: May 3, 2021

Related U.S. Application Data
(60) Provisional application No. 63/019,393, filed on May 3, 2020.

(51) Int. Cl. A61B 7/04; A61B 7/00; A61B 5/00

(52) U.S. Cl. CPC A61B 7/04; A61B 7/003; A61B 5/0004; A61B 2560/0406; A61B 2562/0204; A61B 2562/028; A61B 2562/221; A61B 5/6832

(57) ABSTRACT

One or more auscultation sensors attached to the skin of an at-least-prospectively contagiously-infected patient are connected via a corresponding associated one or more sensor cables so as to provide for one or more health care practitioners to listen to auscultation sounds from the one or more auscultation sensors from a relatively safe distance, without a need for close proximity to the patient when listening.

### CROSS-REFERENCE TO RELATED APPLICATIONS

The instant application claims benefit of U.S. Provisional Application Ser. No. 63/019,393 filed on 3 May 2020, which is incorporated herein by reference in its entirety.

### BRIEF DESCRIPTION OF THE DRAWINGS

In the accompanying drawings:

FIG. 1a illustrates a first aspect of an auscultation system, with four of the auscultation sensors thereof attached to the front side of a torso of a patient;

FIG. 1b illustrates two of the auscultation sensors of the first-aspect auscultation system attached to the back of the patient illustrated in FIG. 1a;

FIG. 2a illustrates the attachment locations of auscultation sensors on the front side of a torso of the patient illustrated in FIG. 1a;

FIG. 2b illustrates the attachment locations of auscultation sensors on the back of the patient illustrated in FIG. 1b;

FIG. 3 illustrates a control unit of the auscultation system illustrated in FIG. 1a connected to a sensor harness-hub via an umbilical cable, and illustrates a block diagram of wire-connected auscultation sensors connected to the sensor harness-hub;

FIG. 4 illustrates the control unit of FIG. 3 operatively coupled to the sensor harness-hub via an umbilical cable, and a plug of a wire-connected auscultation sensor in association with the sensor harness-hub, with the control unit powered by a battery that may be carried in an external battery holster, and further illustrates a pair of earbuds that can plug into the control unit to enable a heath care practitioner to listen to auscultation sounds from auscultation sensors plugged into the sensor harness-hub;

FIG. 5 illustrates a perspective view of an umbilical cable that provides for connecting the sensor harness-hub to the control unit illustrated in FIGS. 3 and 4;

FIG. 6 illustrates an exploded perspective view of the umbilical cable and a plug of a wire-connected auscultation sensor in relation to the associated sensor harness-hub;

FIG. 7 illustrates a rear perspective view of the sensor harness-hub connected with the umbilical cable;

FIG. 8 illustrates a top perspective view of first aspect of an auscultation sensor;

FIG. 9 illustrates a side cross-sectional view of the first aspect auscultation sensor illustrated in FIG. 8;

FIG. 10 illustrates a top perspective view of an adhesive membrane of the first aspect auscultation sensor illustrated in FIGS. 8 and 9;

FIG. 11a illustrates a conceptual side cross-sectional view of a bell-portion of an inverted-bell housing of an auscultation sensor, having a concave parabolic shape;

FIG. 11b illustrates a conceptual side cross-sectional view of a bell-portion of an auscultation sensor, having a convex parabolic shape;

FIG. 12a illustrates a conceptual side cross-sectional view of a bell-portion of an inverted-bell housing of an auscultation sensor, having a concave spherical shape;

FIG. 12b illustrates a conceptual side cross-sectional view of a bell-portion of an inverted-bell housing of an auscultation sensor, having a convex spherical shape;

FIG. 13a illustrates side profile view of a first aspect of a wired auscultation sensor configured for sensing relatively lower-frequency signals;

FIG. 13b illustrates a top perspective view of the first-aspect wired auscultation sensor illustrated in FIG. 13a;

FIG. 14 illustrates a side cross-sectional view of the first-aspect wired auscultation sensor illustrated in FIGS. 13a and 13b;

FIG. 15a illustrates side profile view of a second aspect of a wired auscultation sensor configured for sensing relatively higher-frequency signals;

FIG. 15b illustrates a top perspective view of the second-aspect wired auscultation sensor illustrated in FIG. 15a;

FIG. 16 illustrates a side cross-sectional view of the second-aspect wired auscultation sensor illustrated in FIGS. 15a and 15b;

FIGS. 17a illustrates a side cross-sectional view of a first embodiment of a third aspect of an auscultation sensor;

FIGS. 17b illustrates a bottom plan view of the first-embodiment, third-aspect auscultation sensor illustrated in FIGS. 17a;

FIGS. 18a illustrates a side cross-sectional view of a second embodiment of the third aspect of an auscultation sensor;

FIGS. 18b illustrates a bottom plan view of the second embodiment, third-aspect auscultation sensor illustrated in FIGS. 18a;

FIG. 19a illustrates an isometric view of a Micro-Electro-Mechanical System (MEMS) acoustic transducer, viewed from the sensing side thereof;

FIG. 19b illustrates an isometric view of a Micro-Electro-Mechanical System (MEMS) acoustic transducer, viewed from the housing side thereof;

FIG. 20 illustrates a MEMS acoustic transducer assembly incorporating the Micro-Electro-Mechanical System (MEMS) acoustic transducer illustrated in FIGS. 19a and 19b, prior to its assembly in the associated auscultation sensor;

FIG. 21a illustrates a bottom perspective view of a cap portion of the auscultation sensor illustrated in FIGS. 17a through 18b;

FIG. 21b illustrates a top perspective view of a base portion of the auscultation sensor illustrated in FIGS. 17a through 18b;

FIG. 21c illustrates a top perspective view of the assembled auscultation sensor illustrated in FIGS. 17a through 18b;

FIGS. 22a-c illustrate a first set of side cross-sectional views of a conically-shaped inverted-bell housing of an auscultation sensor for a corresponding variety of different cone angles, configured to cooperate with a first particular model of an associated microphone;

FIGS. 23a-c illustrate a second set of side cross-sectional views of a conically-shaped inverted-bell housing of an auscultation sensor for a corresponding variety of different cone angles, configured to cooperate with a second particular model of an associated microphone;

FIGS. 24a-c illustrate a third set of side cross-sectional views of a conically-shaped inverted-bell housing of an auscultation sensor for a corresponding variety of different cone angles, configured to cooperate with a third particular model of an associated microphone;

FIGS. 25a-c illustrate a fourth set of side cross-sectional views of a conically-shaped inverted-bell housing of an auscultation sensor for a corresponding variety of different cone angles, configured to cooperate with a fourth particular model of an associated microphone;

FIG. 26 illustrates a side view of a B-Lo-F acoustically-shielded low-frequency sensor and an associated electrical cable;

FIG. 27 illustrates an isometric view of the B-Lo-F acoustically-shielded low-frequency sensor illustrated in FIG. 26;

FIG. 28a illustrates an side cross-sectional view of the B-Lo-F acoustically-shielded low-frequency sensor illustrated in FIGS. 26 and 27;

FIG. 28b illustrates an elastomeric cup of an elastomeric shroud incorporated in the B-Lo-F acoustically-shielded low-frequency sensor illustrated in FIGS. 26 through 28a;

FIG. 28c illustrates an elastomeric pad of an elastomeric shroud incorporated in the B-Lo-F acoustically-shielded low-frequency sensor illustrated in FIGS. 26 through 28b;

FIG. 29 illustrates an isometric view of an interface grate;

FIG. 30 illustrates a plan view of the interface grate illustrated in FIG. 29

FIG. 31 illustrates a side view of the interface grate illustrated in FIGS. 29 and 30;

FIG. 32 illustrates a side cross-sectional view of the interface grate illustrated in FIGS. 29 through 31;

FIG. 33 illustrates an exploded view of an adhesive pad assembly that is usable in cooperation with any of auscultation sensors illustrated in FIGS. 13a-18b and 26-28c;

FIG. 34 illustrates a block diagram of first and second aspects of an auscultation system;

FIG. 35 illustrates a block diagram of a first aspect of a control unit of an auscultation system;

FIG. 36 illustrates a block diagram of first and third aspects of an auscultation system; and

FIG. 37 illustrates a block diagram of a second aspect of a control unit of an auscultation system.

### DESCRIPTION OF EMBODIMENT(S)

When confronted with a pandemic caused by a highly infectious respiratory disease, there exists a need for health care professionals (HCPs) to protect themselves from becoming infected by that disease when examining patients who might so afflicted. A conventional stethoscope that would commonly be used to perform auscultation to listen to the heart, lung and abdomen of a prospectively ill patient can require the HCP to be within a sufficiently close range of the patient to make the HCP vulnerable to catching a highly infectious disease for example, a highly infectious respiratory disease—from a patient that turns out to be afflicted therewith.

For example, in the year 2020, the world is presently experiencing a pandemic from the respiratory pathogen SARS-CoV-2, the virus that causes the highly infectious respiratory disease COVID-19, which originated in the year 2019 in China. COVID-19 is highly contagious, and infection therefrom can be easily transmitted to the HCP and other patients, which has put extreme pressure on the health care professionals who are fighting this disease. For example, approximately ⅓ of COVID-19 patients in China, and up to 20 percent of those in the U.S. and Canada, have been reported to be health care workers. Currently there is a shortage of HCPs to deal with this disease, resulting in the recall of retired personnel and even the early graduation of personnel from medical and nursing schools. Due to this shortage of personnel, it is imperative to protect HCPs who are on the front lines of the COVID-19 pandemic and are up to 10 times more likely to be exposed to SARS-CoV-2. This issue may be compounded by the recall of the retired or older HCP workforce—a population that is more vulnerable to the virus—to fill the workforce shortage. HCPs who contract COVID-19 are effectively taken out of this mission critical workforce, and can spread the virus to friends and family and experience significant adverse outcomes such as death and disability. COVID-19 is a major threat to the healthcare workforce globally. Reducing the chance of exposure to COVID-19, to other highly infectious diseases, or to antibiotic-resistant strains of bacteria known as superbugs, is important not only to the HCP but to the well-being of most everyone globally in the international society.

The stethoscope which allows the HCP to listen to the heart, lungs, abdomen, and other anatomical locations is a key component of the physical examination for patients suspected to have the COVID-19 virus. Providers in hospitals, especially on the front lines in Urgent Care, Emergency Room (ER), Intensive Care Unit (ICU), bio-contaminant unit, and radioactive settings, are at high risk for contracting COVID-19. Although the recommended distance for safety is at least six feet, conventional manually-applied stethoscope technology, a critical bedside tool, requires the HCP to be in close proximity (less than 28 inches of conventional stethoscope tubing) to the patient with COVID-19 and increases the risk of person-to-person transmission. Prior literature has shown infectious contamination of the stethoscope diaphragm from contact with the skin of an infected patient. Disinfecting stethoscopes between patients is not standardized or may not be adequate to reduce risk to COVID-19 contamination especially in emergency rooms with heavy patient volume. Many ER doctors are choosing not to perform critical stethoscope examinations due to fear of increased transmission to other patients or to themselves. Prior to the COVID-19 pandemic, research studies by the MAYO Clinic, and many others, have shown that the contamination level of the conventional stethoscope is substantial even after a single physical examination, and can be a main route of infection.

The risk to medical professionals, from self-infection, or transference to another patient or family member, can greatly reduced if auscultation to perform a heart and lung examination occurs at a safe distance of no less than two meters (6.5 feet) from the patient. Furthermore, for patients who are hospitalized after having been diagnosed as having highly infectious respiratory disease, there exists a need for continued auscultation over an extended period of time.

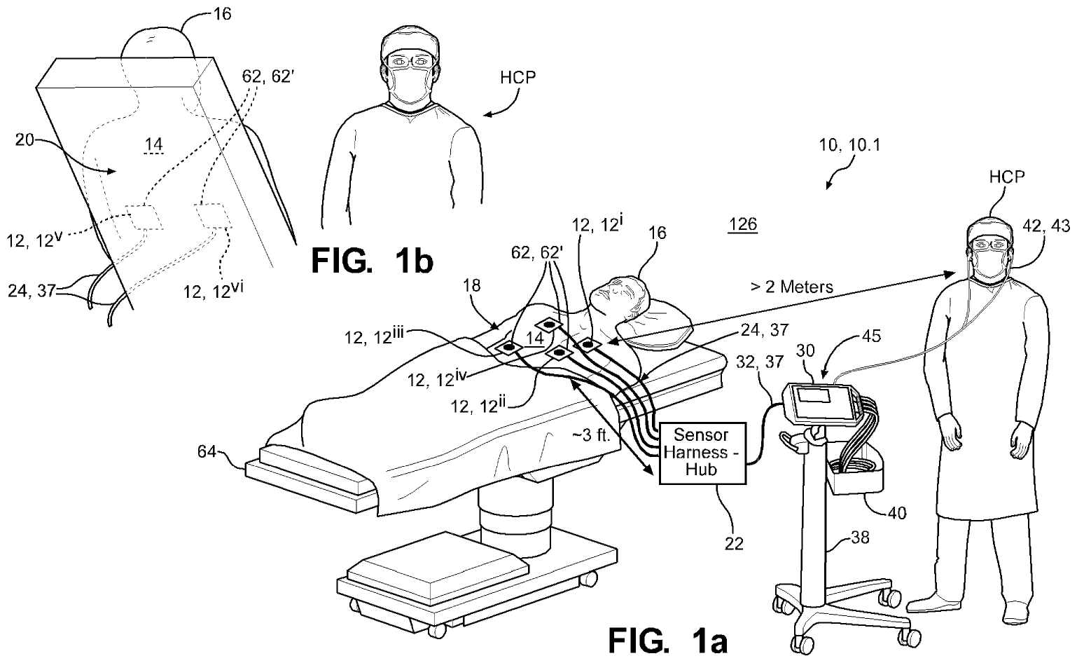

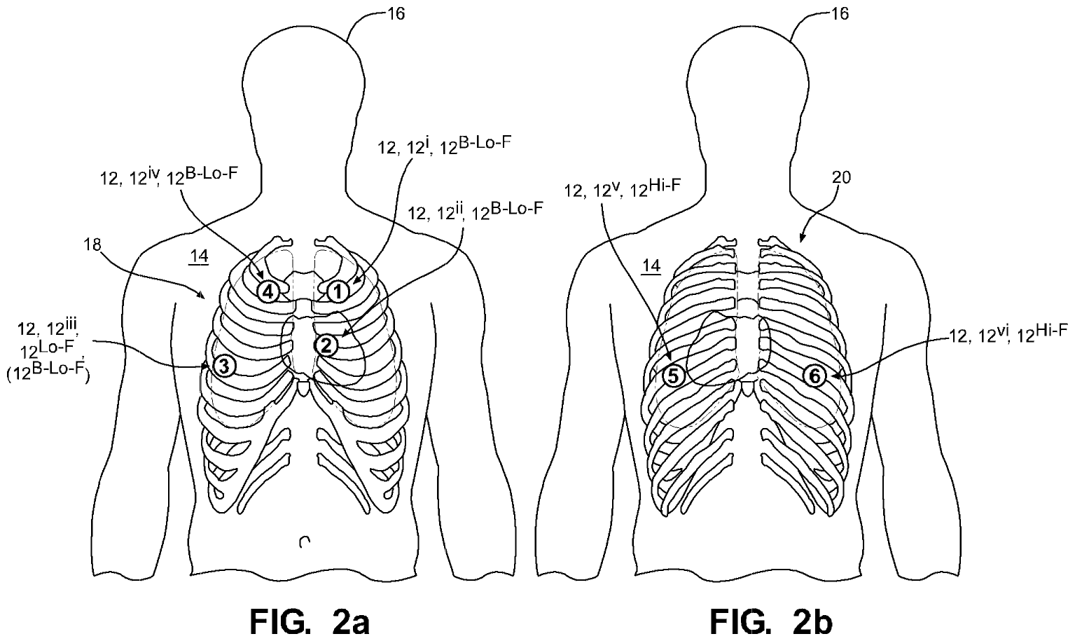

To these ends, referring to FIGS. 1a and 1b, an auscultation system 10 incorporates one or more auscultation sensors 12 that are adhesively attached to the skin 14 of a patient 16. For example, referring also to FIGS. 2a and 2b, in one example of an application of the auscultation system 10, six auscultation sensors 12, 12ⁱ, 12ⁱⁱ, 12ⁱⁱⁱ, 12^{iv}, 12^{v}, 12^{vi} are used simultaneously, four on the front side of the torso 18 of the patient 16, and two on the back 20 of the patient 16, with five of the sensors associated with lung lobes, and the sixth associated with both the heart and a remaining lung lobe.

Referring again to FIG. 1a, in accordance with a first aspect, a protocol for attending to patients 16 with COVID-19—or more generally, patients with a highly-contagious disease, particularly a highly-contagious respiratory disease—is for HCPs within the same room or enclosed space as the patient 16 to be fully protected against infection from the patient 16, for example, by donning infection-resistant gowns or suits, respirators, gloves and possibly face-shields or hoods, for example, as illustrated by the health care practitioner HCP in FIG. 1a who is sufficiently-well protected to provide for safely attaching the illustrated auscultation sensors 12, 12ⁱ, 12ⁱⁱ, 12ⁱⁱⁱ, 12^{iv}, 12^{v}, 12^{vi} to the skin 14 of the patient 16 without becoming exposed to infection from the patient 16.

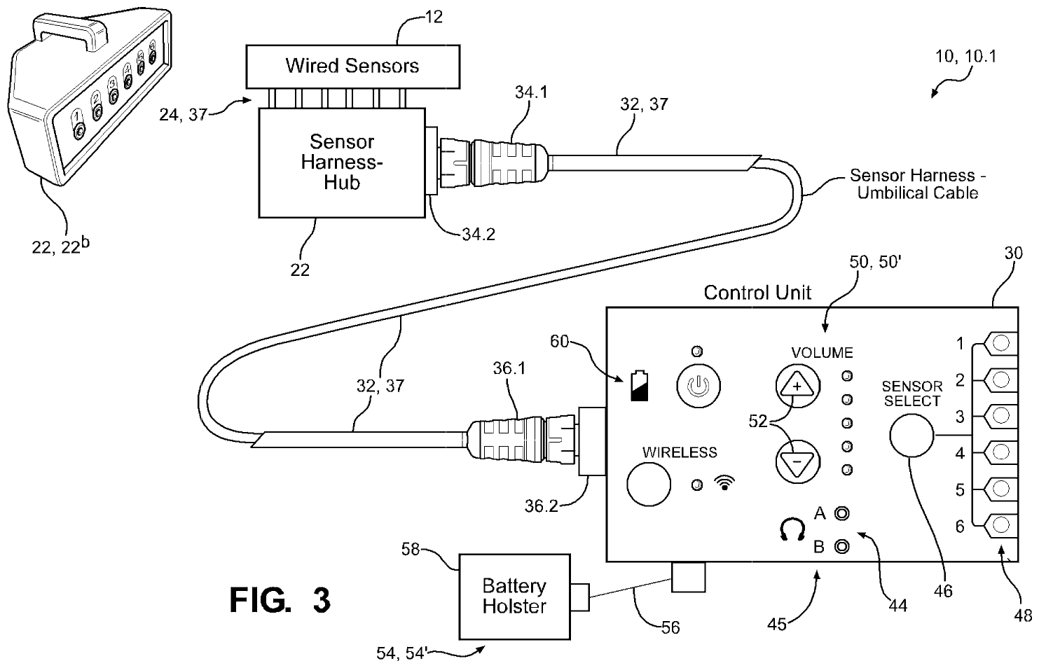

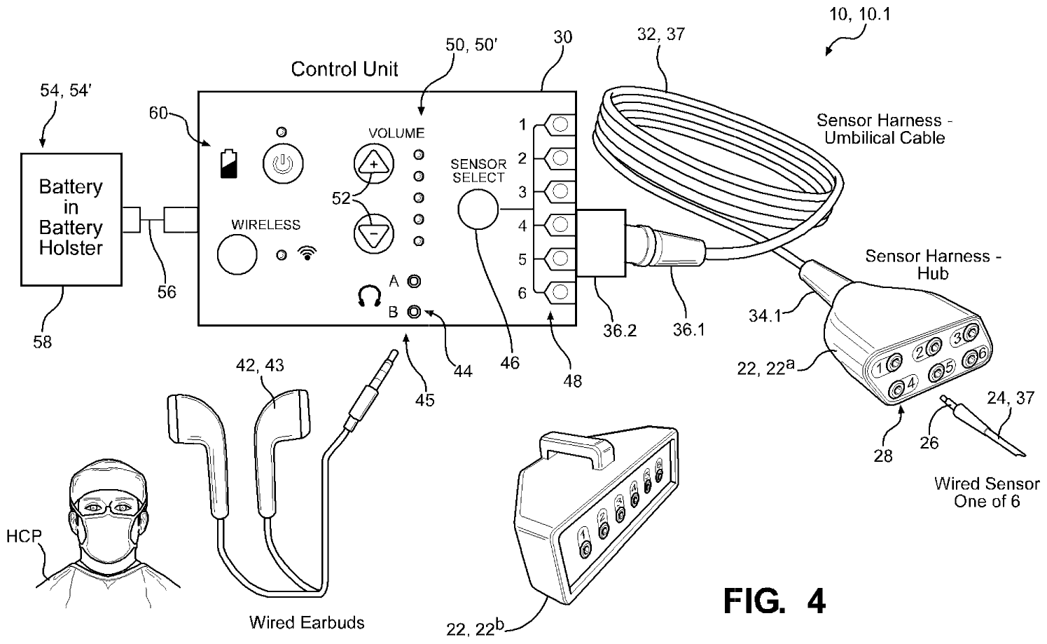

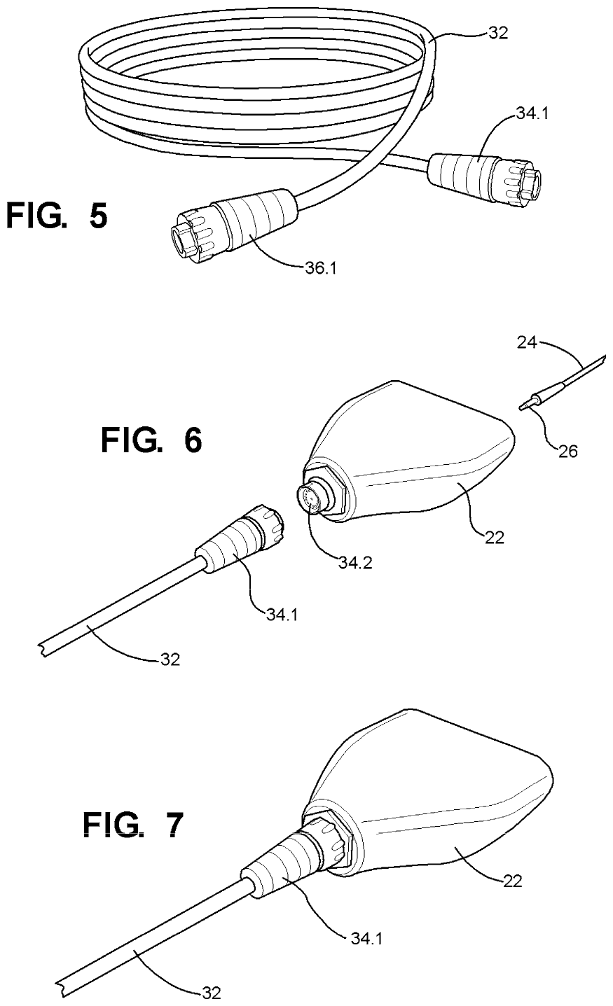

Referring also to FIGS. 3 through 7, each of the auscultation sensors 12, 12ⁱ, 12ⁱⁱ, 12ⁱⁱⁱ, 12^{iv}, 12^{v}, 12^{vi} is wire-connected to a sensor harness-hub 22 by a corresponding sensor wire-cable 24 that is terminated with a plug 26 that plugs into a corresponding socket 28 on the sensor harness-hub 22. In one set of embodiments, the sensor harness-hub 22 is configured with six sockets 28, each of which provides for receiving a plug 26 of a corresponding auscultation sensor 12, so as to provide for auscultation at six corresponding locations on the patient 16. For example, FIG. 4 illustrates a first embodiment of a sensor harness-hub 22, 22a for which six sockets 28 are organized in two rows of three sockets 28, and FIGS. 3 and 4 illustrate a second embodiment sensor harness-hub 22, 22^{b} for which the six in-line sockets 28 in a single row. The sensor harness-hub 22 is in turn connected to a control unit 30 via an associated sensor harness-umbilical-cable 32 of cleanable, medical-grade construction, the latter of which is removably coupled to both the sensor harness-hub 22 and the control unit 30 with corresponding connectors 34.1, 36.1 at respective ends of the sensor harness-umbilical-cable 32, that mate with corresponding mating connectors 34.2, 36.2 on the sensor harness-hub 22 and the control unit 30, respectively, so as to provide for coupling a corresponding auscultation signal 37 from each corresponding auscultation sensor 12 to the control unit 30, wherein each corresponding auscultation signal 37 is responsive to internal sounds-or-vibrations from within the body of the patient 16 that propagate therewithin to the corresponding location of the corresponding auscultation sensor 12 on the surface of the skin 14 of the patient 16.

In accordance with a second aspect of a protocol for attending to patients 16 with COVID-19, or a similarly highly-contagious disease, medical paraphernalia—for example, the auscultation sensors 12, 12ⁱ, 12ⁱⁱ, 12ⁱⁱⁱ, 12^{iv}, 12^{v}, 12^{vi} and associated sensor wire-cables 24—that can either come in contact with, or become in close proximity to, the patient 16, is preferably economically constructed so as to be discardable after a single use with an at-least-prospectively contagiously-infected patient 16, so as to mitigate against contamination of either the associated health care practitioners HCP, or the associated hospital room or objects therein, from prospectively contaminated hardware after removal from the patient 16. Furthermore, relatively-more-expensive medical hardware for example, the sensor harness-hub 22, the sensor harness-umbilical-cable 32 and the control unit 30 in one set of embodiments, are located at least about 1 meter (3 feet) from the patient 16, and are constructed so as to be cleanable either by wipe-down, or by exposure to biologic cleaning agents such as ozone or ultra-violet light for example, in satisfaction of the requirements for cleaning in accordance with IEC60601. For example, in one set of embodiments, the sensor harness-umbilical-cable 32 is up to 3 meters in length. Alternatively, the sensor harness-hub 22 having surfaces that would be susceptible to contact when connecting the auscultation sensors 12, 12ⁱ, 12ⁱⁱ, 12ⁱⁱⁱ, 12^{iv}, 12^{v}, 12^{vi} thereto—may also be discardable after a single use with an at-least-prospectively contagiously-infected patient 16.

Furthermore, the portions of the elements of the auscultation system 10 with which the health care practitioner HCP would interact when monitoring the patient 16 are configured to be located at least about 2 meters from the patient 16 so as to further reduce the likelihood of transmitting infection from the patient 16 to the health care practitioner HCP. Accordingly, in one set of embodiments, the control unit 30 is mounted at a location that is, or can be, at a distance from the patient 16 that is sufficiently great for example, in one set of embodiments, at least 3 meters (10 ft.)—to prevent transmission of disease to a health care practitioner HCP who wishes to safely examine the patient 16. For example, in one set of embodiments, the control unit 30 is attached to a wheeled pole 38 which has a basket 40 for temporarily storing the sensor wire-cables 24 e.g. coiled,—for example, either when not in use, or when in use during conditions when contagious infection is not a risk so that the control unit 30 can then be used in relatively close proximity to the patient 16. Alternatively, the control unit 30 could be fixedly mounted at a location either inside or outside the same room or space as the patient 16 at a distance from the patient 16 that is sufficiently great to prevent transmission of disease to an associated health care practitioner HCP. Yet further alternatively, in cooperation with below-described wireless embodiments of the control unit 30 for which the health care practitioner HCP need not be close to the control unit 30 during operation thereof, the control unit 30 could be mounted at any location within reception of associated wireless signals.

In accordance with one mode of operation, the health care practitioner HCP can plug a set of headphones, external speakers, or earbuds 42 i.e. a listening device 43 incorporating one or more associated electroacoustic transducers—into a socket 44 on the control unit 30 acting as an associated communications node 45, so as to provide for listening to sound from a selected one of the auscultation sensors 12, 12ⁱ, 12ⁱⁱ, 12ⁱⁱⁱ, 12^{iv}, 12^{v}, 12^{vi}, which is selected by progressively depressing a sensor-select touch-switch 46 until an indicator light 48 corresponding to the desired auscultation sensor 12, 12ⁱ, 12ⁱⁱ, 12ⁱⁱⁱ, 12^{iv}, 12^{v}, 12^{vi} is illuminated, wherein each associated electroacoustic transducer generates a sound responsive to an electrical auscultation signal 37 from the corresponding selected auscultation sensor 12, 12ⁱ, 12ⁱⁱ, 12ⁱⁱⁱ, 12^{iv}, 12^{v}, 12^{vi}. Although earbuds 43, 42 are explicitly illustrated in the accompanying drawings, it should be understood that these could be substituted with any type of plug-in listening device incorporating an associated one or more electroacoustic transducers, for example, two electroacoustic transducers that might be associated with stereo earbuds 43, 42 or stereo headphones. For example, in one set of embodiments, the earbuds 43, 42 are discardable after a single use wth an at-least-prospectively contagiously-infected patient 16 to as to reduce the risk of transmission of disease to a health care practitioner HCP. The control unit 30 further incorporates a signal strength indicator 50—for example, either a column of LED indicator lights 50′ as illustrated, or a plurality of progressively longer light-bars, the illuminated length of which indicates signal strength—that indicate the strength of the audio signal for the selected auscultation sensor 12, 12ⁱ, 12ⁱⁱ, 12ⁱⁱⁱ, 12^{iv}, 12^{v}, 12^{vi}, which can be adjusted up or down by depressing a corresponding volume-adjustment touch-switches 52. In one set of embodiments, the control unit 30 is powered from a battery 54, for example, an externally-mounted battery 54′,—for example, that is operatively coupled to the control unit 30 with an associated power cable 56 and which is mounted in a battery holster 58—and incorporates a battery-state-of-charge indicator 60 to provide an indication responsive to the state-of-charge of the associated battery 54. Alternatively, the battery 54—either rechargeable or not—could be located within the control unit 30, and an internal rechargeable battery, if used, could be charged with either a plug-in or an inductively-coupled charger.

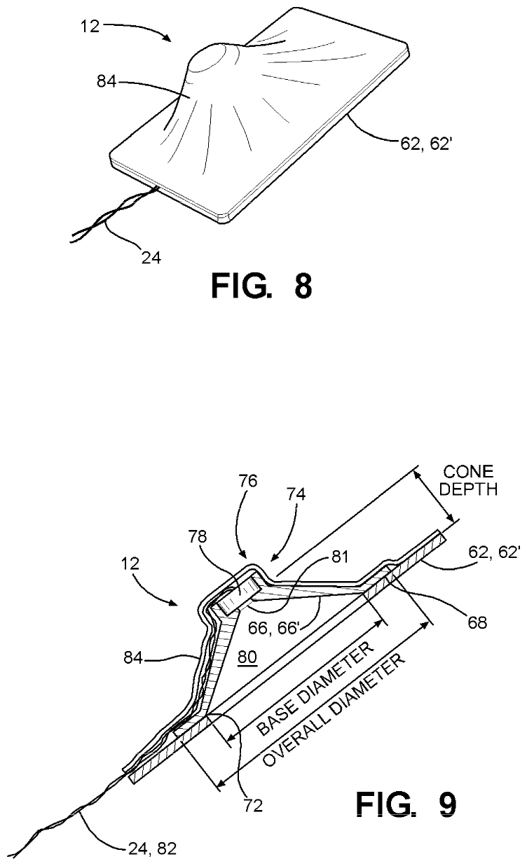

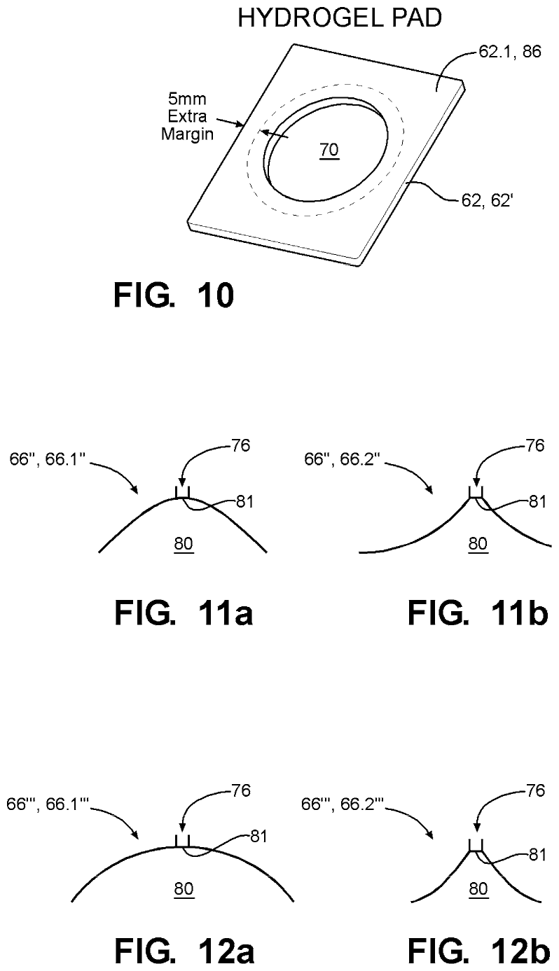

Referring also to FIGS. 8-10, in operation, each of the six auscultation sensors 12, 12ⁱ, 12ⁱⁱ, 12ⁱⁱⁱ, 12^{iv}, 12^{v}, 12^{vi} can be adhesively attached to the patient 16 by a health care practitioner HCP, for example, by a nurse, who would be fully suited and protected from exposure to the infectious agent, with the adhesive attachment made using a single-use self-adhesive membrane 62 satisfying the skin-safe requirements of IEC60601, for example, a hydrogel pad 62, 62′, between the base of the auscultation sensor 12, 12ⁱ, 12ⁱⁱ, 12ⁱⁱⁱ, 12^{iv}, 12^{v}, 12^{vi} and the skin 14 of the patient 16, and with the auscultation sensors 12, 12ⁱ, 12ⁱⁱ, 12ⁱⁱⁱ, 12^{iv}, 12^{v}, 12^{vi} located at standard locations for heart and lung examination. For example, in accordance with one set of practices, single-use auscultation sensors 12, 12ⁱ, 12ⁱⁱ, 12ⁱⁱⁱ, 12^{iv}, 12^{v}, 12^{vi} are placed on the skin 14 of the patient 16 during admittance to the hospital and secured with custom hydrogel pads 62, 62′, wherein the auscultation sensors 12, 12ⁱ, 12ⁱⁱ, 12ⁱⁱⁱ, 12^{iv}, 12^{v}, 12^{vi} are each then connected via a corresponding single-use sensor wire-cable 24 to a sensor harness-hub 22, for example, the latter of which in one set of embodiments is removably attached to convenient location, such as to the frame of the bed 64 upon which the patient 16 is located. Thereafter, with the sensor harness-hub 22 connected to the control unit 30 and the latter positioned at a safe distance from the patient 16, a health care practitioner HCP can then listen touch free—for example via single-use earbuds 43, 42 that are plugged into the control unit 30—to heart or lung sounds in real time, and via the control unit 30, selecting which auscultation sensor 12, 12ⁱ, 12ⁱⁱ, 12ⁱⁱⁱ, 12^{iv}, 12^{v}, 12^{vi} they wish to listen to, and adjusting the level of sound volume thereof using the associated volume-adjustment touch-switches 52, while in the same room as the patient 16, but without being in direct contact with the patient 16. The auscultation sensors 12, 12ⁱ, 12ⁱⁱ, 12ⁱⁱⁱ, 12^{iv}, 12^{v}, 12^{vi} can later be moved, in association with the application of additional associated self-adhesive membranes 62, 62′, to provide for examining additional auscultation sites of choice. Once placed, the sensors can remain in place for an extended period of time—for example, for at least 24 hours and up to several days—so as to provide for touch free auscultation at any time without direct patient contact by the health care practitioner HCP, including both listening on-demand in real time by the health care practitioner HCP, or by machine recording as described hereinbelow.

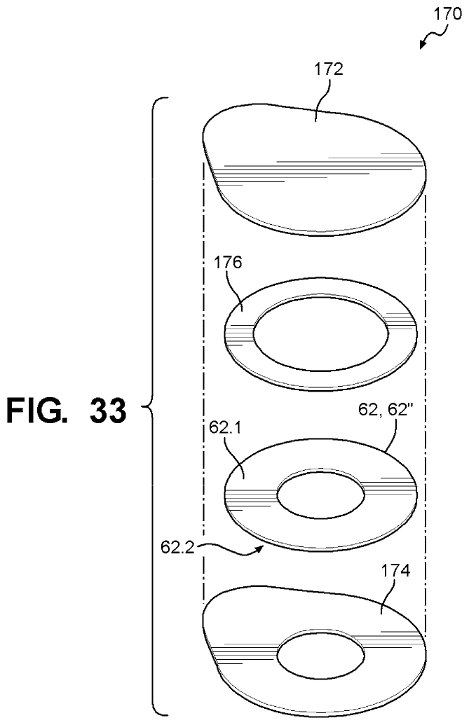

Each auscultation sensor 12, 12ⁱ, 12ⁱⁱ, 12ⁱⁱⁱ, 12^{iv}, 12^{v}, 12^{vi} incorporates an inverted-bell housing 66—for example, in one set of embodiments, conically-shaped 66′—with a substantially-planar annular rim 68 that is configured to adhesively attach to the skin 14 of the patient 16—i.e. to the outer surface of the skin 14—using an associated hydrogel pad 62, 62′, the latter of which incorporates a hole 70 that is intended to be aligned with the mouth opening 72 of the annular rim 68. For example, in one set of embodiments, the hydrogel pad 62, 62′ is about 50 mm square, with a 30 mm diameter hole 70. Alternatively, the inverted-bell housing 66 may have a modified conical shape with a tapered-cylindrical mouth opening abutting a conical inner surface, for example, as illustrated in FIG. 14, 16 or 28a, in cooperation with a annular hydrogel pad 62, 62′, for example, as illustrated in FIG. 33. Generally, the shape of the inverted-bell housing 66 is not limiting. The apex 74 of the inverted-bell housing 66 incorporates a receptacle 76 to receive a microphone 78 (or more generally, an acoustic transducer 78), the latter of which provides for sensing sound from within the cavity 80 of the inverted-bell housing 66 through an associated acoustic port 81 at the apex 74 of the inverted-bell housing 66. Conductive leads 82 of, or operatively coupled to, the sensor wire-cable 24 are operatively coupled to the microphone 78 to provide power thereto from the control unit 30 acoustic port (if necessary for a particular microphone 78), and to transmit an audio signal therefrom to the control unit 30. Optionally, the outside of the inverted-bell housing 66 and, if exposed, the microphone 78, together with the associated conductive leads 82 extending from the microphone 78, may be covered with a membrane 84 for example, comprising an elastomeric material—that is sealed to the peripheral portion 86 of the top side 62.1 of the hydrogel pad 62, 62′ outside the annular rim 68 of the inverted-bell housing 66, so as to provide for protecting the auscultation sensor 12, 12ⁱ, 12ⁱⁱ, 12ⁱⁱⁱ, 12^{iv}, and to provide for helping to insulate the cavity 80 of the inverted-bell housing 66 from external acoustic noise.

The shape of the inverted-bell housing 66 and associated cavity 80 is not limiting. For example, referring to FIGS. 11a and 11b, as an alternative to a conically-shaped 66′ inverted-bell housing 66, the inverted-bell housing 66 could be parabolically-shaped 66″, with either a corresponding concave-parabolic profile 66.1″ or a convex-parabolic profile 66.2″, respectively, wherein the concavity and convexity are with respect to the associated cavity 80.

Furthermore, as another example, referring to FIGS. 12a and 12b, as a further alternative to a conically-shaped 66′ inverted-bell housing 66, the inverted-bell housing 66 could be spherically-shaped 66′″, with either a corresponding concave-spherical profile 66.1′″ or a convex-spherical profile 66.2″, respectively, wherein the concavity and convexity are with respect to the associated cavity 80.

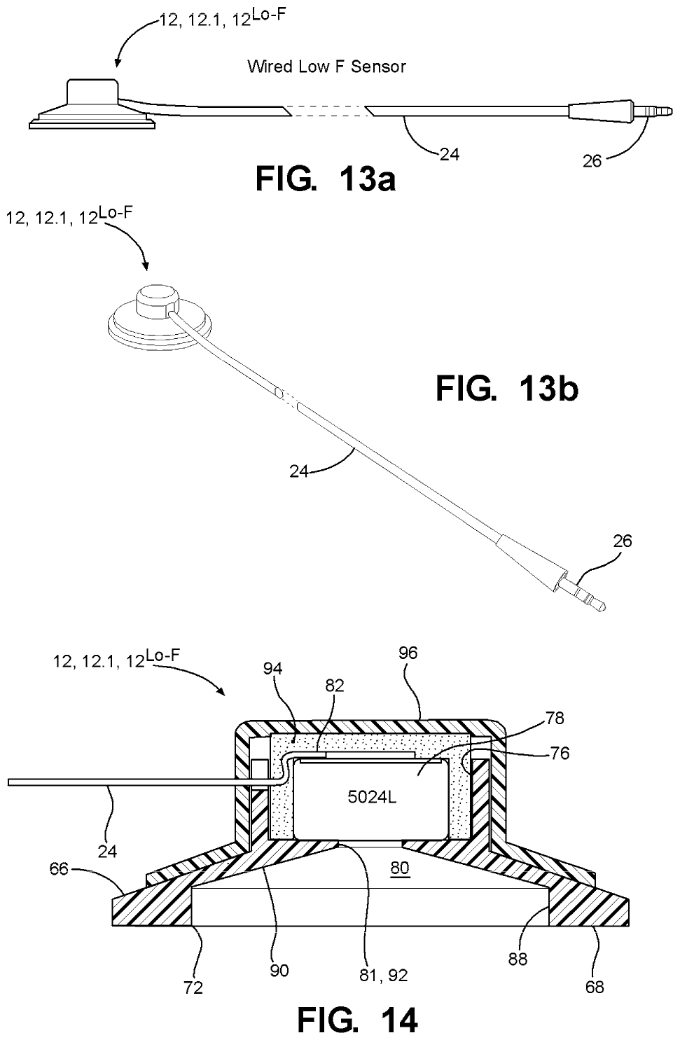

Referring to FIGS. 8, 9, 13a-13b and 14, in what is referred to as a Lo-F auscultation sensor 12, 12^{Hi-F}, a first aspect 12.1 of an auscultation sensor 12, 12.1 incorporates a relatively-higher profile inverted-bell housing 66 that is suitable for sensing relatively lower-frequency sounds, such as cardiac sounds or abdominal sounds from the chest or the abdomen of the patient 16. The illustrated embodiment of the first aspect auscultation sensor 12, 12.1 incorporates a Model AOM-5024L microphone 78 that is available from PUT Audio Inc. of Dayton, Ohio The cavity 80 of the inverted-bell housing 66—having an overall depth of about 5 millimeters comprises cylindrical portion 88 that is interposed between the mouth opening 72 of the cavity 80 and a conical portion 90 that leads into an acoustic port 81/orifice 92 through which sound waves communicate with the microphone 78, wherein the cylindrical 88 and conical 90 portions each span about half the depth of the cavity 80. An elastomeric cap 94—extending over the back of the microphone 78 and around the sides thereof provides for at least partially insulating the microphone 78 from background sounds. The elastomeric cap 94 and microphone 78 are retained within a receptacle 76 on the back side of the inverted-bell housing 66 by a cap 96 that is bonded to a portion of the outside surface of the inverted-bell housing 66. The mouth opening 72 is surrounded by an annular rim 68 for example, in one set of embodiments, having a 5 millimeter radial extent that provides for bonding to the top side 62.1 of the hydrogel pad 62, 62′ that bonds to the skin 14 of the patient 16.

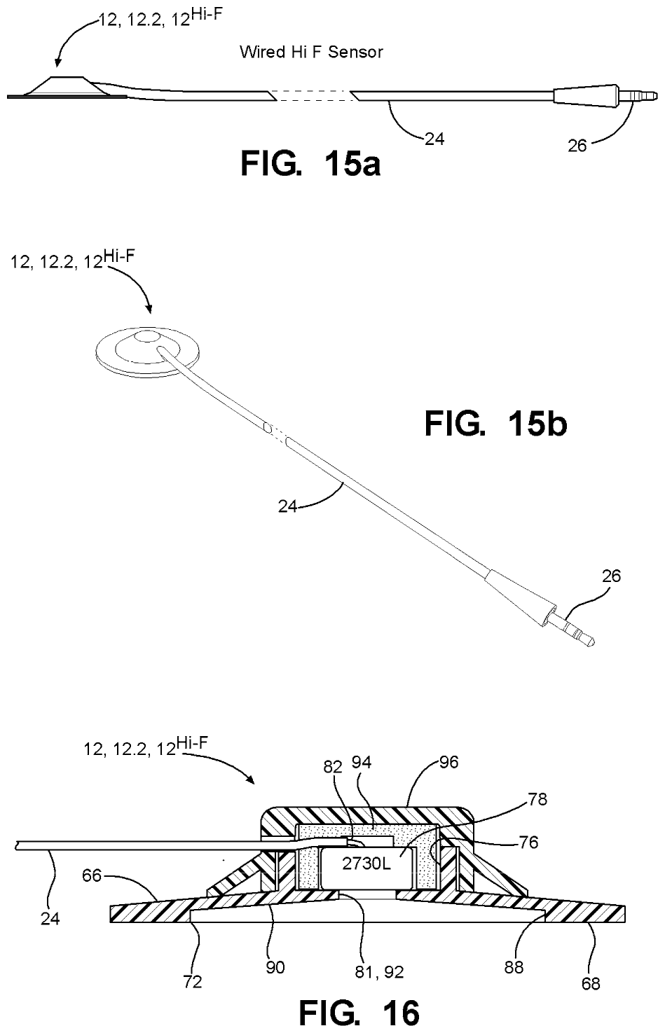

Referring to FIGS. 15a-15b and 16, in what is referred to as a Hi-F auscultation sensor 12, 12^{Hi-F}, a second aspect 12.2 of an auscultation sensor 12, 12.2 incorporates a relatively-lower profile inverted-bell housing 66 that is suitable for sensing relatively higher-frequency sounds, such as lung sounds or heart sounds from the back of the patient 16. The illustrated embodiment of the first aspect auscultation sensor 12, 12.1 incorporates a Model POM-2730L microphone 78 that is available from PUI Audio Inc. of Dayton, Ohio. The cavity 80 of the inverted-bell housing 66—having an overall depth of about 1.5 millimeters comprises cylindrical portion 88 that is interposed between the mouth opening 72 of the cavity 80 and a conical portion 90 that leads into an acoustic port 81/orifice 92 through which sound waves communicate with the microphone 78, with the cylindrical 88 and conical 90 portions each spanning about half the depth of the cavity 80. An elastomeric cap 94—extending over the back of the microphone 78 and around the sides thereof provides for at least partially insulating the microphone 78 from background sounds. The elastomeric cap 94 and microphone 78 are retained within a receptacle 76 on the back side of the inverted-bell housing 66 by a cap 96 that is bonded to a portion of the outside surface of the inverted-bell housing 66. The mouth opening 72 is surrounded by an annular rim 68 for example, in one set of embodiments, having a 5 millimeter radial extent that provides for bonding to the top side 62.1 of the hydrogel pad 62, 62′ that bonds to the skin 14 of the patient 16.

For example, in one set of embodiments, the inverted-bell housings 66 of the first 12.1 and second 12.2 aspect auscultation sensors 12, 12.1, 12.2 may be formed of plastic, for example, by either injection molding or 3-D printing. For example, in one set of embodiments, the inverted-bell housing 66 and the cap 96 of the first 12.1 and second 12.2 aspect auscultation sensors 12 are each constructed of injection-molded for example, simultaneously-injection-molded depending from a common sprue—ABS plastic.

In each of the above-illustrated embodiments of the first 12.1 and second 12.2 aspect auscultation sensors 12, 12.1, 12.2, and of particular relevance, the second aspect auscultation sensor 12, 12.2, the mouth opening 72 and the cavity 80 of the inverted-bell housing 66 are each free of internal structure, so as to be entirely exposed to the skin 14 of the patient 16. With the auscultation sensors 12, 12ⁱ, 12ⁱⁱ, 12ⁱⁱⁱ, 12^{iv}, 12^{v}, 12^{vi} attached to the skin 14 of the patient 16 on both the front side of the torso 18 and the back 20 of the patient 16, and with the patient 16 lying on a bed 64, at least one of the auscultation sensors 12, 12ⁱ, 12ⁱⁱ, 12ⁱⁱⁱ, 12^{iv}, 12^{v}, 12^{vi} will likely become sandwiched between the patient 16 and the bed 64. For at least the auscultation sensors 12, 12ⁱ, 12ⁱⁱ, 12ⁱⁱⁱ, 12^{iv}, 12^{v}, 12^{vi} upon which the patient 16 might lie, an auscultation sensor 12 having a relatively lower profile and a relatively higher aspect ratio will be relatively more comfortable to the patient 16 than an auscultation sensor 12 having a relatively higher profile and a relatively lower aspect ratio. However, for some patients 16, the cavity 80 of the inverted-bell housing 66 of a relatively lower profile, higher aspect-ratio (width/height ratio) auscultation sensor 12 is relatively more susceptible to being plugged by the skin 14 of the patient 16 extending thereinto so as to at least partially conform to the internal surface thereof, as a result of the patient 16 lying on that auscultation sensor 12, which can result in a substantial attenuation of the associated acoustic signal from the auscultation sensor 12.

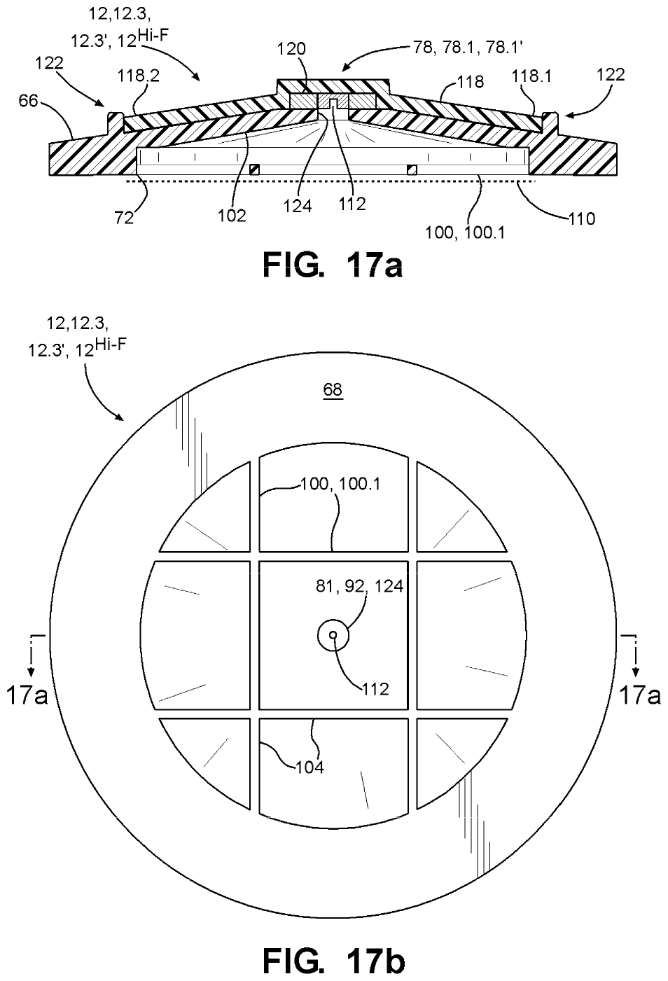

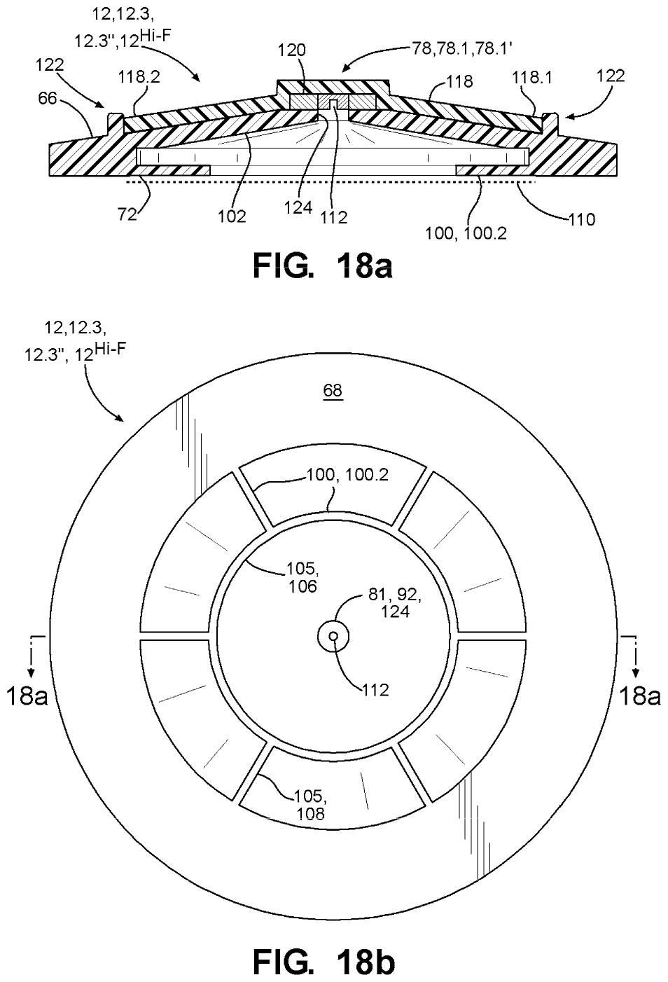

Referring to FIGS. 17a-18b, in accordance with a third aspect 12.3 of an auscultation sensor 12, 12.3, the mouth opening 72 of the inverted-bell housing 66 incorporates a grate 100 thereacross that provides for preventing the skin 14 of the patient 16 from contacting the surface 102 of the cavity 80 of the inverted-bell housing 66, which would otherwise cause an attenuation of the sound waves being sensed by the microphone 78. For example, referring to

FIGS. 17a-17b, in accordance with a first embodiment of the third aspect auscultation sensor 12, 12.3′, the grate 100.1 extends along a rectilinear grid 104. As another example, referring to FIGS. 18a-18b, in accordance with a second embodiment of a third aspect auscultation sensor 12, 12.3″, the grate 100.2 extends along a polar grid 105, for example, comprising a circular ring 106 connected to the mouth opening 72 of the inverted-bell housing 66 with a plurality of radial spokes 108 extending radially outwards from the circular ring 106. The third aspect auscultation sensor 12, 12.3′, 12.3″ may optionally incorporate a mesh layer 110 on the outside of the grate 100, 100.1, 100.2 that provides for distributing the force of the grate 100, 100.1, 100.2 over the skin 14 of the patient 16 and thereby mitigate against irritation from the grate 100, 100.1, 100.2 that might otherwise result from the long-term use of the third aspect auscultation sensor 12, 12.3′, 12.3″. It should be understood that the inverted-bell housing 66 absent the grate 100, 100.1, 100.2, in cooperation with the associated microphone 78, would function as a second aspect auscultation sensor 12, 12.2, which would be suitable if intrusion of the skin 14 of the patient 16 into the cavity 80 of the inverted-bell housing 66 was not problematic.

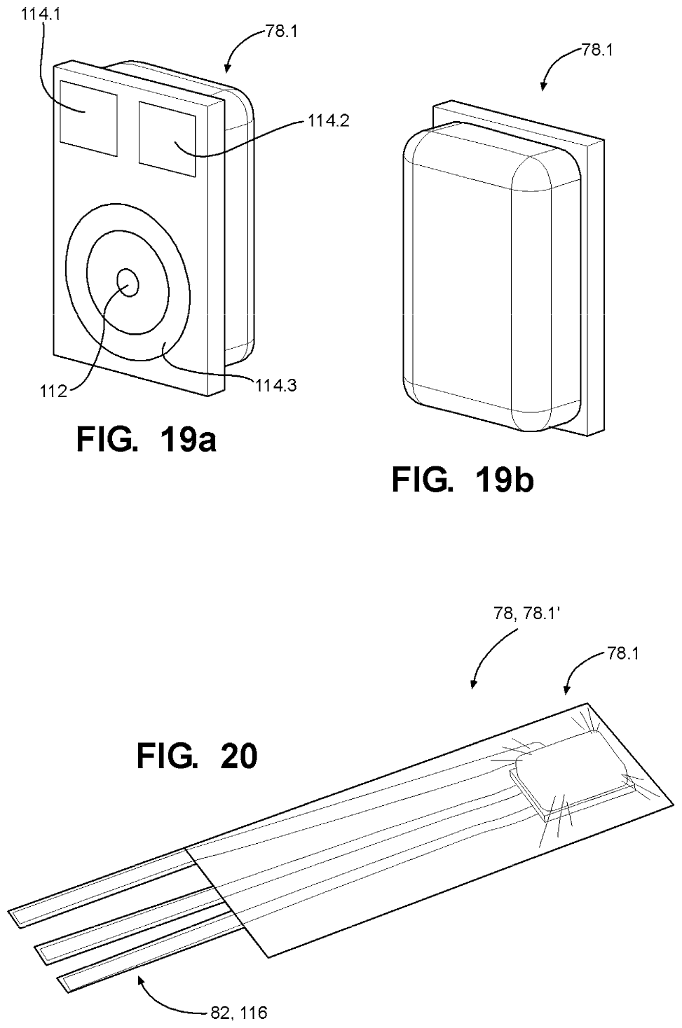

Referring to FIGS. 19a-b, in accordance with one set of embodiments, the microphone 78 comprises a Micro-Electro-Mechanical System (MEMS) acoustic transducer 78.1—for example, in one embodiment, a model AMM-2738-B-R microphone from PUT Audio, Inc. of Dayton, Ohio with an associated acoustic port-hole 112 that provides for receiving the sound to be transduced, and that incorporates power 114.1, signal-output 114.2, and ground 114.3 terminals. Referring to FIG. 20, in one embodiment, the power 114.1, signal-output 114.2, and ground 114.3 terminals of the MEMS acoustic transducer 78.1 are operatively coupled to a plurality of foil conductors 116, with the MEMS acoustic transducer 78.1 and foil conductors 116 encapsulated within layers of Kapton® tape to form an associated MEMS acoustic transducer assembly 78.1′ which is used as the microphone 78, 78.1′ of the auscultation sensor 12, 12′, 12″.

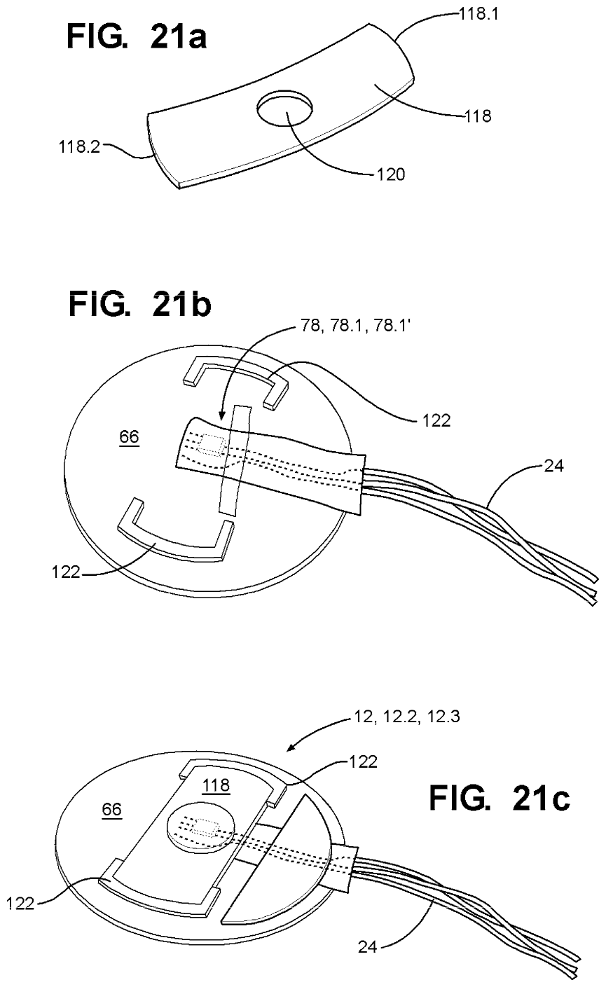

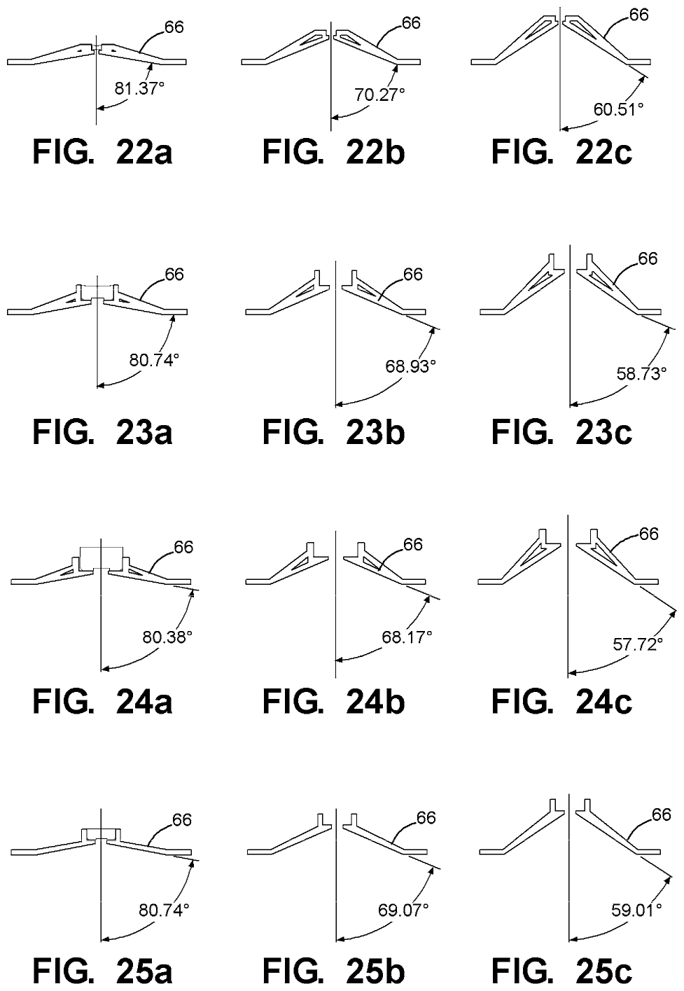

Referring to FIGS. 17a-18b and 21a-21c, in one set of embodiments, the microphone 78, 78.1′ is sandwiched between the outside of the inverted-bell housing 66—at the apex 74 thereof—and a cap 118 that incorporates a recess 120 to receive the MEMS acoustic transducer 78.1 of the microphone 78, 78.1′. The ends 118.1, 118.2 of the cap 118 cooperate with corresponding socket portions 122 on the outside of the inverted-bell housing 66, so as to provide for retaining and aligning the microphone 78, 78.1′ relative to the inverted-bell housing 66. For example, in one set of embodiments, the ends 118.1, 118.2 of the cap 118 snap into the socket portions 122 on the outside of the inverted-bell housing 66. Alternatively, or additionally, the cap 118 may be either bonded, welded or secured with one or more fasteners to the outside of the inverted-bell housing 66. The inverted-bell housing 66 incorporates an acoustic port 124 at the apex 74 thereof that is aligned with an associated acoustic port-hole 112 of the associated MEMS acoustic transducer 78.1. For example, referring to FIG. 21b, in one set of embodiments, prior to assembly of the cap 118 on the outside of the inverted-bell housing 66, after aligning the acoustic port-hole 112 of the associated MEMS acoustic transducer 78.1 with the acoustic port 124 of the inverted-bell housing 66, the relative alignment therebetween is maintained by taping the MEMS acoustic transducer assembly 78.1′ to the outside of the inverted-bell housing 66. Referring to FIGS. 22a through 25c, the inverted-bell housing 66 can be configured to cooperate with a variety of different microphones 78—illustrated examples of which are available from PUT Audio Inc. of Dayton, Ohio—with a variety of different conical profiles and associated cone angles. For example, FIGS. 22a-c illustrate a plurality of different inverted-bell housings 66 of successively higher profile and lower aspect ratio, for cooperation with a model # AMM-2738-B-R MEMS acoustic transducer assembly 78.1′ used as the associated microphones 78. As another example, FIGS. 23a-c illustrate a plurality of different inverted-bell housings 66 of successively higher profile and lower aspect ratio, for cooperation with a model # POW-2242L-C3310-B-R microphone 78. As yet another example, FIG. 24a-c illustrate a plurality of different inverted-bell housings 66 of successively higher profile and lower aspect ratio, for cooperation with a model # AOM-5024L-HD-R microphone 78. As yet another example, FIG. 25a-c illustrate a plurality of different inverted-bell housings 66 of successively higher profile and lower aspect ratio, for cooperation with a model # ROM-2235P-HD-R microphone 78.

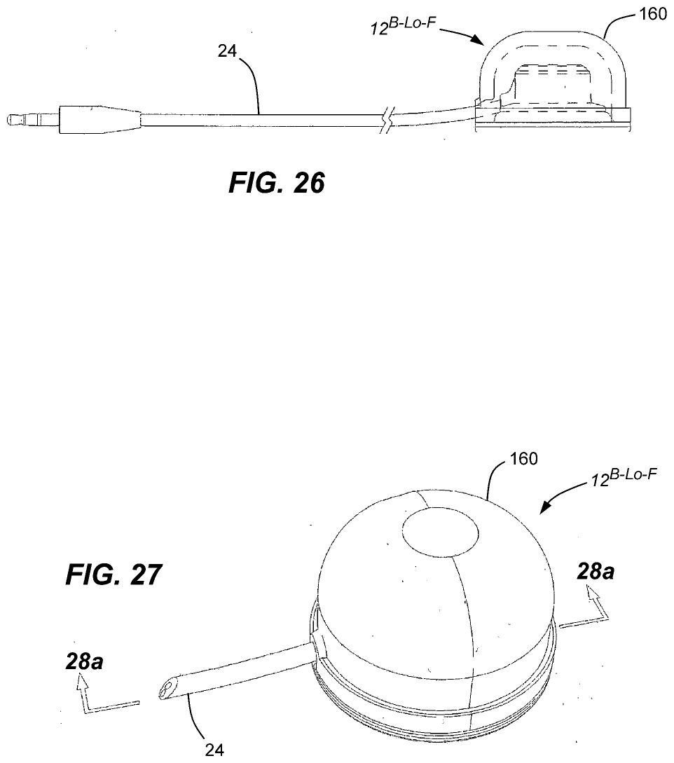

Referring to FIGS. 26-28a, in what is referred to as a B-Lo-F auscultation sensor 12, 12B-Lo-F a variant of the Lo-F first aspect auscultation sensor 12, 12″^{-F} illustrated in FIG. 14,—configured to provide for sensing relatively-lower-frequency sounds, incorporates a domed cap 160, for example, shaped like a bowler hat, so as to provide for rejecting or attenuating background acoustic interference. For example, in one set of embodiments, the domed cap 160 is 3-D printed with PETG (Polyethylene terephthalate glycol) plastic to form a 3 mm thick shell, which is then bonded for example, using cyano-acrylate glue, e.g. Loctite® 4011—to the upper surface of the associated annular rim 68 of the inverted-bell housing 66 of the underlying Lo-F auscultation sensor 12, 12.1, leaving an air gap 162 between the outside surface of the inverted-bell housing 66 and the inside surface of the domed cap 160.

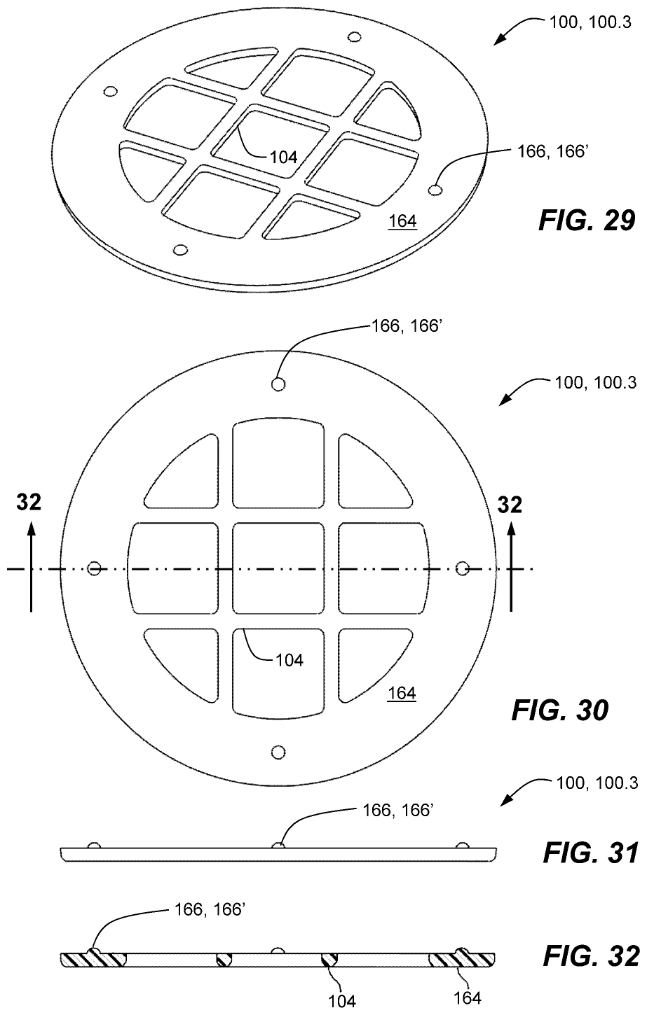

Referring also to FIGS. 29-32, the base (i.e. patient-facing surface) of the annular rim 68 of the B-Lo-F auscultation sensor 12, 12^{B-Lo-F} is bonded to a peripheral annular-ring portion 164 of an interface grate 100, 100.3, for example, using cyano-acrylate glue, e.g. Loctite® 4011, the same as used to bond the cap 96 to the inverted-bell housing 66, wherein the inner diameter of the peripheral annular-ring portion 164 is substantially the same as that of the mouth opening 72 of the inverted-bell housing 66. The interface grate 100, 100.3 incorporates a rectilinear grid 104, for example, with the outside edge corners rounded so as to not irritate the skin 14 of the patient 16. The peripheral annular-ring portion 164 incorporates a plurality of dimples 166, e.g. hemispherical dimples 166′—for example, uniformly radially positioned and equi-angularly spaced around the peripheral annular-ring portion 164—that provide for mating with corresponding dimple sockets 168 on the base of the annular rim 68 that are sufficiently large to accommodate the dimples 166, 166′, that provide for the peripheral annular-ring portion 164 to abut the base of the annular rim 68, and that provide for aligning the interface grate 100, 100.3 with the annular rim 68 of the inverted-bell housing 66. As another example, in another set of embodiments, the inverted-bell housing 66, the cap 96, and the interface grate 100, 100.3 are each constructed of injection-molded for example, simultaneously-injection-molded depending from a common sprue—ABS plastic.

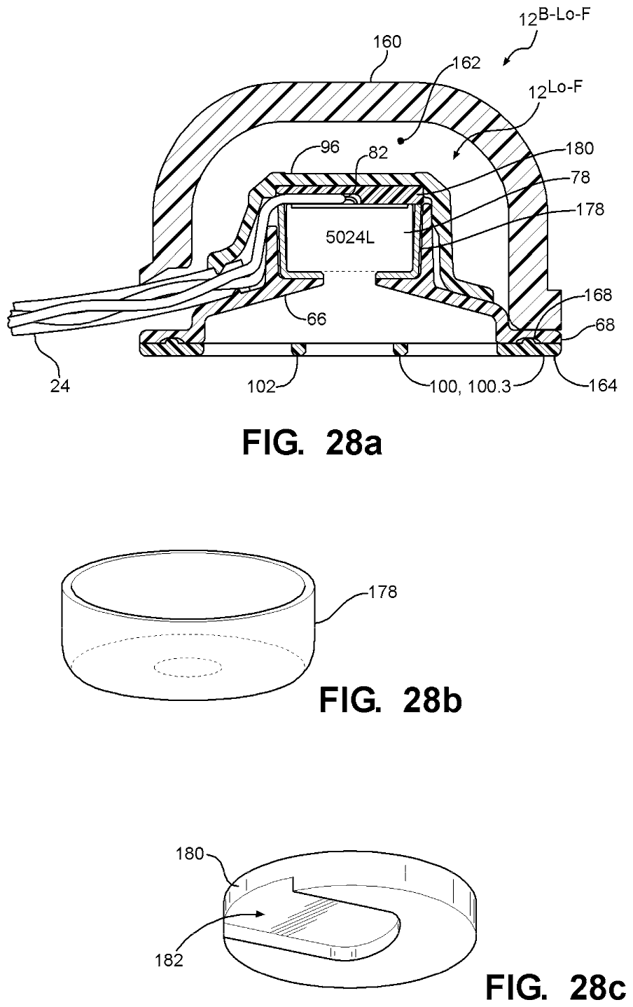

Referring also to FIGS. 28b and 28c, the B-Lo-F auscultation sensor 12, 12^{B-Lo-F} further incorporates an elastomeric shroud around the associated microphone 78 for example, in the form of a cup 178 (also referred to as a “sock”) abutting the base and side-wall of the microphone 78, and a pad 180 abutting the top of the microphone 78, so as to provide for acoustically isolating the microphone 78 from the receptacle 76 of the inverted-bell housing 66 and from the cap 96, so as to provided for dampening vibrations of the microphone 78 therewithin responsive to patient-induced motion of the inverted-bell housing 66, and to provide for further acoustically insulating the microphone 78 from external noise. The underside of the pad 180 incorporates a recess 182 to provide clearance for the associated conductive leads 82 that attach to the associated microphone 78. For example, in one set of embodiments, the cup 178 and the pad 180 are injection molded for example, simultaneously injection molded depending from a common sprue—of 30 Duro-A elastomeric TPU (Thermoplastic Polyurethane), for example, Santoprene®. Similarly, an elastomeric shroud provided by a cup 178 and a pad 180 can also used in either the Lo-F auscultation sensor 12, 12^{Lo-F} or the Hi-F auscultation sensor 12, 12^{Hi-F}, instead of the elastomeric cap 94 illustrated in FIGS. 14 and 16.

Referring to FIG. 33, an adhesive pad assembly 170 that provides for attaching an auscultation sensor 12 to the skin 14 of the patient 16 incorporates an annular hydrogel pad 62, 62″ sandwiched between a top release liner 172 and a bottom liner 174, further incorporating an annular intermediate liner 176 between the top side 62.1 of the annular hydrogel pad 62, 62″ and the top release liner 172 having the same outer diameter as that of the annular hydrogel pad 62, 62″, the latter of which is larger than that of the annular rim 68 of the inverted-bell housing 66. The inner diameter of the annular intermediate liner 176 is substantially the same as the outer diameter of the annular rim 68 of the inverted-bell housing 66. The inner diameter of the annular hydrogel pad 62, 62″ is substantially the same as the mouth opening 72 of the inverted-bell housing 66. The annular hydrogel pad 62, 62″ is configured so that the top side 62.1 thereof is intended to attach to the annular rim 68 of the inverted-bell housing 66 after removal of the top release liner 172, wherein the bottom side 62.2 of the annular hydrogel pad 62, 62″ is intended to attach to the skin 14 of the patient 16 after removal of the bottom liner 174. For example, in one set of embodiments, the annular hydrogel pad 62, 62″ comprises KM 40C Hydrogel Long-Term-Wear Skin Adhesive which is rated for at least 24 hours and up to 5-7 days of attachment, and which is oriented with a relatively-stronger-bonding surface on the side to which the auscultation sensor 12 is attached. Furthermore, in one set of embodiments, the bottom 174 and annular intermediate 176 liners are each 3-mil thick, of a different color than the top release liner 172 so as to provide for distinguishing the different sides of the annular hydrogel pad 62, 62″ having different levels of bonding strength.

During use of the adhesive pad assembly 170 to attach an auscultation sensor 12 to the skin 14 of the patient 16, in accordance with one approach, the bottom liner 174 is removed first to provide for attaching the adhesive pad assembly 170 to the skin 14 of the patient 16 at the intended sensing location. Then, the top release liner 172 is removed to provide for attaching the annular rim 68 of the inverted-bell housing 66 to the top side 62.1 of the annular hydrogel pad 62, 62″ within the inner diameter of the annular intermediate liner 176, the latter of which remains in place to prevent clothing or bedding from attaching to the top side 62.1 of the annular hydrogel pad 62, 62″.

Referring again to FIGS. 2a and 2b, in accordance with one embodiment relatively high-frequency-response, Hi-F auscultation sensors 12, 12^{Hi-F}, for example, as illustrated in FIGS. 15a-15b, but with an interface grate as illustrated in FIGS. 29-32, or alternatively, as illustrated in either FIGS. 17a-^{b} or 18a-b, are used as auscultation sensors 12^{v}, 12^{vi} on the back 20 of the patient 16 at the corresponding locations 5, 6 indicated in FIG. 2b, so as to provide for sensing lung sounds which would typically span a relatively higher range of frequencies than cardiac sounds; relatively low-frequency-response sensors with a “bowler-hat” background-noise barrier, i.e. B-Lo-F auscultation sensors 12, 12^{B-Lo-F} example, as illustrated in FIGS. 26-28, are used as auscultation sensors 12ⁱ, 12ⁱⁱ, 12^{iv} on the front side of the torso 18 of the patient 16 at the corresponding locations 1, 2, 4 indicated in FIG. 2a; and a relatively low-frequency-response Lo-F auscultation sensor 12, 12^{Lo-F}—for example, as illustrated in FIG. 14, but with an interface grate as illustrated in FIGS. 29-32, or alternatively, as illustrated in either FIGS. 17a-b or 18a-b, is used as the third auscultation sensor 121ⁱⁱ on the front side of the torso 18 of the patient 16 at the corresponding location 3 indicated in FIG. 2a. Alternatively, a B-Lo-F auscultation sensor 12, 12^{B-Lo-F} could be substituted for the third auscultation sensor 121ⁱⁱ if the position of the corresponding location 3 is moved out of the arm-pit area so as to no be susceptible getting swiped off by movement of the associated arm by the patient 16. Generally, the auscultation sensors 12, 12ⁱ, 12ⁱⁱ, 12ⁱⁱⁱ, 121^{iv}, 12^{v}, 12^{vi} are intended to “listen” to physiologic biosounds and therefore, can be placed at the discretion of a physician on body locations where biosounds are produced. The Lo-F auscultation sensor 12, 12^{Lo-F} and B-Lo-F auscultation sensor 12, 12^{B-Lo-F} provide for better sensitivity in the frequency range of relatively-lower frequency cardiac sounds. Even though the Hi-F auscultation sensors 12, 12^{Hi-F} are capable of sensing the relatively-lower frequencies, relatively-lower frequency components in the signal therefrom are typically filtered out using a software filter. The frequency bandwidth for both types of sensors is in the range of 20 Hz to 1 KHz, but with different internal filtering for the Hi-F auscultation sensors 12, 12^{Hi-F} that are used on the back 20, the latter of which are relatively thinner and therefore have a relatively-lower sensitivity.

In accordance with a first aspect 10.1, the auscultation system 10, 10.1 is operated directly from the control unit 30 that is used as an associated communications node 45 by the associated health care practitioner HCP, for example, within the room 126 within which the patient 16 is located.

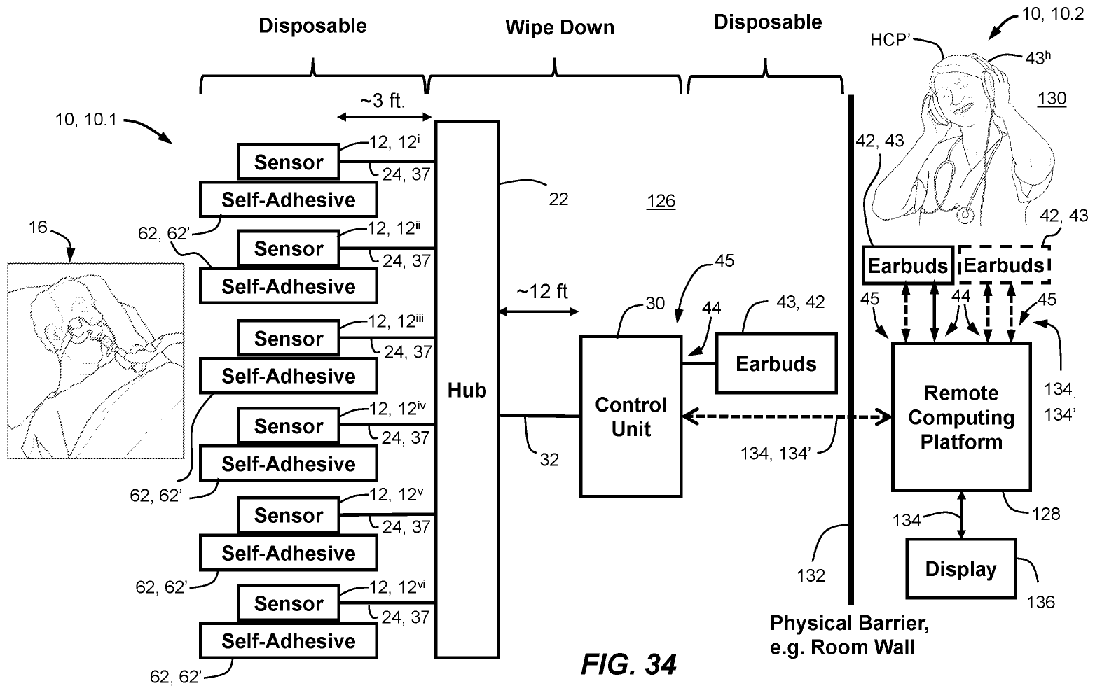

Referring to FIG. 34, in accordance with a second aspect 10.2 of the auscultation system 10, 10.2, the control unit 30 may be paired with an associated remote computing platform 128 to provide for a relatively-remote access—for example, from a relatively-safe location 130, for example, from outside a physical barrier 132 of the room 126 within which the patient 16 is situated—to the auscultation signals 134 associated with heart and lung sounds of the patient 16 that are generated by the auscultation sensors 12, 12ⁱ, 12ⁱⁱ, 12ⁱⁱⁱ, 12^{iv}, 12^{v}, 12^{vi} so as to provide for a remotely-located health care practitioner HCP′ to listen to or observe, the auscultation signals 134 from the auscultation sensors 12, 12ⁱ, 12ⁱⁱ, 12ⁱⁱⁱ, 12^{iv}, 12^{v}, 12^{vi} from outside the patient's room 126 without need for wearing Personal Protective Equipment (PPE) that would otherwise be required to protect the health care practitioner HCP from infection if they were to enter the patient's room 126. For example, the remote computing platform 128 may transit to one or more sets of earbuds 43, 42 or headphones 43, 43^{h}—either wired or wireless,—each associated with a corresponding communications node 45 accessed by a different remotely-located health care practitioner HCP′, so as to provide for one or more remotely-located health care practitioners HCP′ to listen to selected auscultation sound 134′, for example, in one set of embodiments, together with a provision for different remotely-located health care practitioners HCP′ to select the same or different auscultation sounds 134′ to be played on different earbuds 43, 42 or headphones 43, 43^{h}. In accordance with one set of embodiments, multiple remotely-located health care practitioners HCP′ can plug into the remote computing platform 128 and, with a switch, or switches physical or virtual (i.e. software controlled) select the auscultation sites that provides the sound(s) being listened to. Furthermore, in one set of embodiment, the remote computing platform 128 may be configured to store either, or both, the associated auscultation signals 134 or other signals that are sensed by the control unit 30 and transmitted therefrom to the remote computing platform 128, and/or to provide for displaying associated images (e.g. oscillographic-style images) of the received signals on an associated display 136, either in real time, from stored versions thereof, or from a combination of real-time and stored signals. Furthermore, in some embodiments, the remote computing platform 128 may be configured—for example, interfaced with an external communications network, e.g. the internet—as an access point for tele-medicine.

Accordingly, the provision for controlling the control unit 30 from, and for playing auscultation sounds 134′ at, a relatively-safe location 130 provides for conserving valuable Personal Protective Equipment (PPE) resources, and improving cost and resource utilization of Personal Protective Equipment (PPE). Access to the patient's auscultation signals 134 also provides for maximizing the working time of the physician or other health care practitioner HCP if the remotely-located health care practitioner HCP′ is located outside the infection control zone, by reducing or eliminating time needed to install and subsequently remove and dispose Personal Protective Equipment (PPE). Access to the patient's auscultation signals 134 by a remotely-located health care practitioner HCP′ also provides for reducing the risk of spreading infection to other patients from the patient 16 being monitored, by reducing contact of health care practitioners HCP with infectious or potentially infectious patients 16 from whom the infection might otherwise be spread by the health care practitioner HCP.

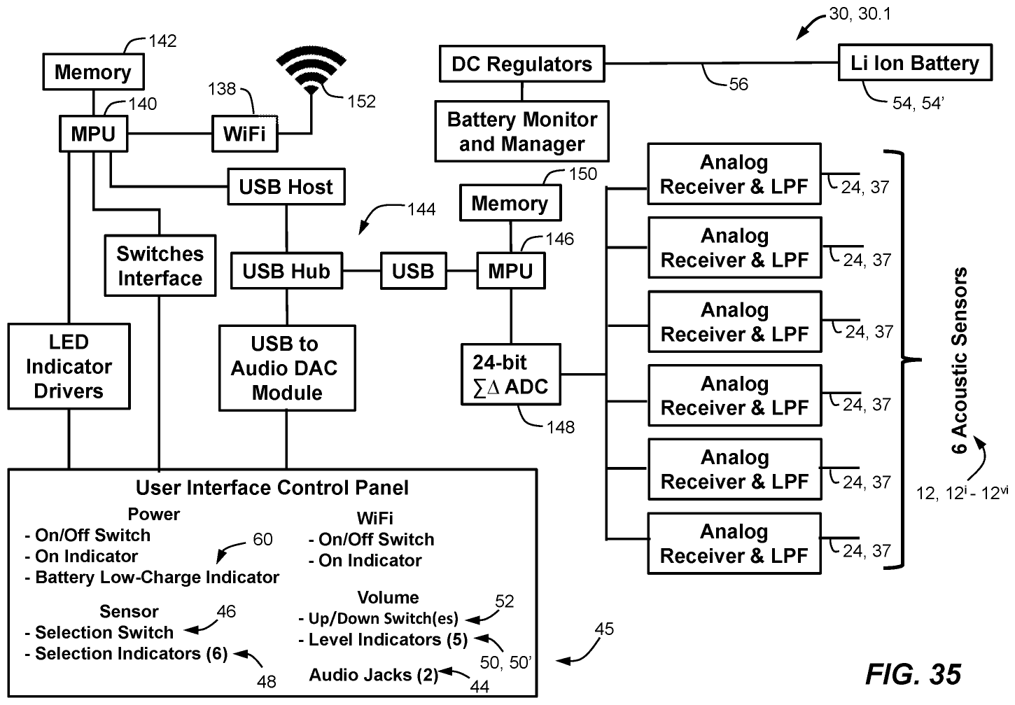

Although the remote computing platform 128 could potentially be wired to the control unit 30, in one set of embodiments, for the sake of convenience and flexibility, the remote computing platform 128 can be implemented with any general purpose computing platform that is WiFi accessible, for example, including, but not limited to a smart-phone, tablet computer, a laptop computer, or a desktop computer, so as to provide for wirelessly communicating with the control unit 30. More particularly, referring to FIG. 35, in accordance with a first aspect 30.1, the control unit 30, 30.1 incorporates a WiFi interface 138 that is operatively coupled to an executive Micro-Processor Unit 140—in cooperation with associated memory 142—that communicates via a Universal Serial Bus (USB) 144 with a local microcontroller 146, the latter of which provides for receiving auscultation signals 134 from each of up to six auscultation sensors 12, 12ⁱ, 12ⁱⁱ, 12ⁱⁱⁱ, 12^{iv}, 12^{v}, 12^{vi}, wherein an analog output from each of the auscultation sensors 12, 12ⁱ, 12ⁱⁱ, 12ⁱⁱⁱ, 12^{iv}, 12^{v}, 12^{vi} is amplified and filtered by an associated front-end receiver and low-pass filter LPF, and then converted to digital form by an associated sigma-delta analog-to-digital filter 148 under control of the local microcontroller 146 in cooperation with associated memory 150.

Referring again to FIG. 35, in operation, the remote computing platform 128 provides for the remotely-located health care practitioner HCP′ to select which of the auscultation sensors 12, 12ⁱ, 12ⁱⁱ, 12ⁱⁱⁱ, 12^{iv}, 12^{v}, 12^{vi} to monitor; to listen to the associated auscultation sound 134′ therefrom via either headphones, earbuds, speakers; to control the gain of the auscultation sound 134′ or auscultation signal 134; or, for some embodiments to view the associated auscultation signal 134, or a transformation thereof, on an associated display 136. In accordance with one set of embodiments, the remote computing platform 128 provides for recording the associated auscultation signals 134—for example, as way or mp3 files—that can be transmitted and subsequently listened to by one or more doctors. Furthermore, in one set of embodiments, both the control unit 30 and the remote computing platform 128 are configured so that the remote computing platform 128 can provide for controlling—via the WiFi interface 138—all of the control functions that are provided for directly by or from the control unit 30 itself.

Accordingly, returning to FIG. 35, upon receipt of wireless request from the remote computing platform 128 for the auscultation signal 134 from a particular auscultation sensor 12, 12ⁱ, 12ⁱⁱ, 12ⁱⁱⁱ, 12^{iv}, 12^{v}, 12^{vi}, the WiFi interface 138 communicates that request to the executive Micro-Processor Unit 140, which in turn interrogates—via the Universal Serial Bus (USB) 144—the local microcontroller 146, the latter of which channels—via the Universal Serial Bus (USB) 144—the selected auscultation signal 134 in real time to the executive Micro-Processor Unit 140 for transmission to the remote computing platform 128, via the WiFi interface 138 and an associated WiFi antenna 152.

The control unit 30 and associated battery 54 provide for sufficient WiFi power, and sufficient physical space, for a WiFi antenna 152 of sufficient gain, to provide for sufficient wireless range over a sufficiently long period of time to accommodate a sufficiently-remotely located remote computing platform 128 so that the remotely-located health care practitioner HCP′ can safely listen to the associated auscultation sounds 134′, or view the associated auscultation signals 134, without risk of infection if not otherwise protected by Personal Protective Equipment (PPE), while also reducing the need for relatively proximally-close interactions of associated health care practitioners HCP with an infectious patient 16.

The control unit 30 may be additionally configured to interface with other patient sensors, for example, but not limited to, one or more of an ECG sensor, a fingertip SPO2 sensor, a blood-pressure sensor, or one or more temperature sensors, the data from which may then be transmitted to the remote computing platform 128 for either display thereon, or recording thereby.

In one set of embodiments, the control unit 30 either incorporates, or interfaces with, an ambient noise sensor, for example, so as to provide for automatic cancellation of associated ambient noise within the auscultation signals 134 during heart or lung auscultation.

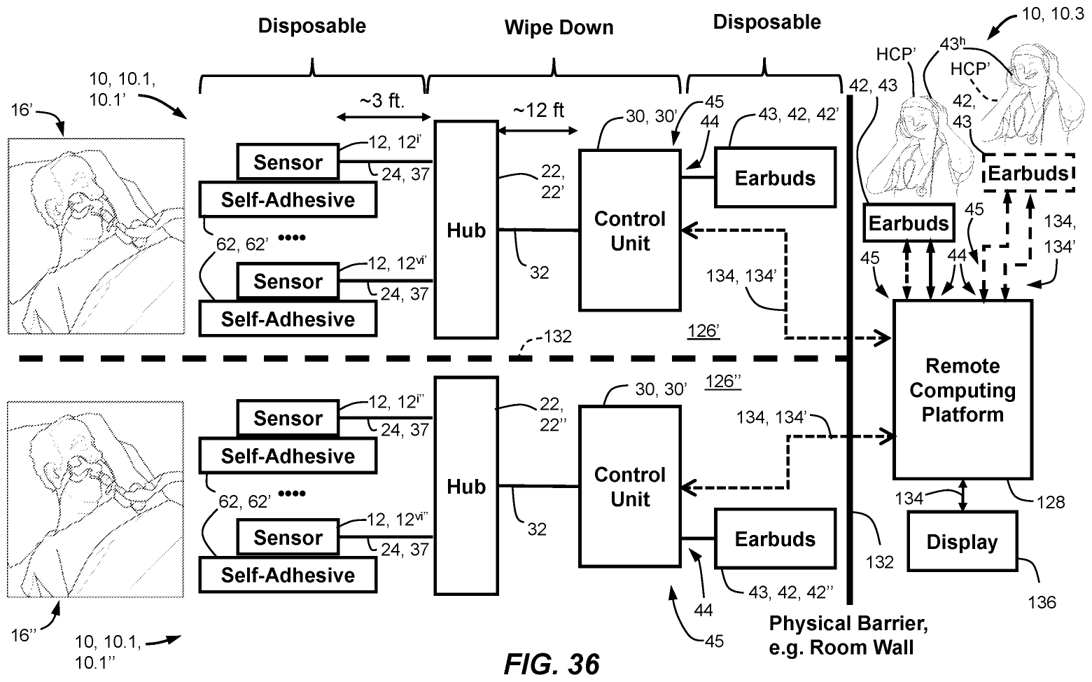

Referring to FIG. 36, in accordance with a third aspect 10.3 of the auscultation system 10, 10.3, the third-aspect auscultation system 10, 10.3 is the same as the above-described second-aspect auscultation system 10, 10.2 except for providing for the functionality thereof for each of a plurality of patients 16, 16′, 16″, each of which is associated with a corresponding first-aspect auscultation system 10, 10.1′, 10.1″, for example, wherein a first patient 16′—possibly in a first room 126′—associated with a first set of auscultation sensors 12, 12^{i′}, . . . , 12^{vi′} operatively coupled to a first control unit 30, 30′ via a first sensor harness-hub 22, 22′, can be locally monitored from an associated communications node 45 using a first set of earbuds 43, 42, 42′, and wherein a second patient 16″—possibly in a second room 126″—associated with a second set of auscultation sensors 12, 12^{i″}, . . . , 12^{vi″} operatively coupled to a second control unit 30, 30″ via a second sensor harness-hub 22, 22″, can be locally monitored from an associated communications node 45 using a second set of earbuds 43, 42, 42″, and wherein both the first 30′ and second 30″ control units 30 are in communication with the same remote computing platform 128, the latter of which provides for selectively accessing and controlling either of the associated control units 30, 30′, 30″, so that one or more remotely-located health care practitioner HCP′ can select—for listening or display from an associated communications node 45—auscultation sounds 134′ from any of the associated auscultation sensors 12 of either the associated first set of auscultation sensors 12, 12ⁱ, . . . , 12^{vi′} or the associated second set of auscultation sensors 12, 12^{i″}, . . . , 12^{vi″}, without either touching, or being in the same space or spaces as either of the patients 16, 16′, 16″.

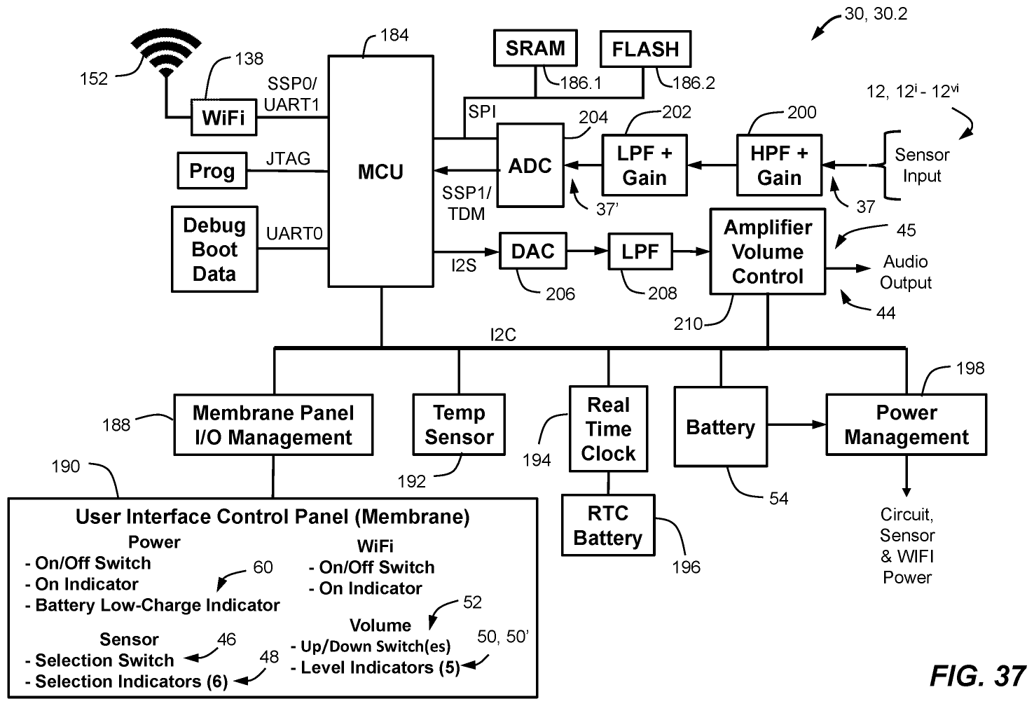

Referring to FIG. 37, a second aspect 30.2, the control unit 30, 30.2 does not incorporate a local microcontroller 146 as does the first aspect control unit 30, 30.1, but instead incorporates a single Micro-Controller Unit (MCU) 184 that provides for directly processing signal-conditioned auscultation signals 37′. For example, in one set of embodiments, the Micro-Controller Unit (MCU) 184 contains two cores—an ARM Cortex-M4 processor and an ARM Cortex-MO processor—that can cooperate with a variety of on-chip memory, including Static Random-Access memory (SRAM) 186.1, FLASH memory 186.2, EEPROM, ROM or One-Time Programmable (OTP) memory, for example via a Serial Peripheral Interface (SPI) bus. For example, in one embodiment, the second aspect control unit 30, 30.2 incorporates two 512 Kbyte SRAM chips 186.1 that provide for storing 24-bit data from six auscultation sensors 12, 12ⁱ, 12ⁱⁱ, 12ⁱⁱⁱ, 12^{iv}, 12^{v}, 12^{vi} sampled at 4 KHz over a period of 14.6 seconds, and two 64 Mbyte FLASH memory 186.2 chips that provide for storing 24-bit data from six auscultation sensors 12, 12ⁱ, 12ⁱⁱ, 12ⁱⁱⁱ, 12^{iv}, 12^{v}, 12^{vi} sampled at 4 KHz over a period of 31 minutes seconds. The Micro-Controller Unit (MCU) 184 utilizes an I2C bus to communicate with a membrane panel interface 188 that cooperates with an associated membrane-switch-based user-interface control panel 190 that functions the same as that described hereinabove in conjunction with the first aspect control unit 30, 30.1, to provide for actuating associated LED indicators and to provide for detecting when associated membrane-switch buttons are pressed, for example, power the system on or off, to adjust the listening volume, to select the sensor channel for listening, and to toggle WIFI communications. The

Micro-Controller Unit (MCU) 184 also utilizes the I2C bus additional control and monitoring functions, including 1) to monitor the temperature of an associated temperature sensor 192 located in a region of the associated printed circuit board (PCB) where most of the heat is generated; 2) to read a real-time clock 194 that is powered with a coin battery 196; 3) to monitor the status of a rechargeable battery 54 within the second aspect control unit 30, 30.2 that provides power to the associated circuitry and the WIFI interface 138 of the second aspect control unit 30, 30.2, and provides power to the associated auscultation sensors 12, 12ⁱ, 12ⁱⁱ, 12ⁱⁱⁱ, 12^{iv}, 12^{v}, 12^{vi}. An associate power management module 198 utilizes a first DC/DC converter to provide power to the WIFI interface 138, and a second DC/DC converter to provide power to the remaining circuitry and to the auscultation sensors 12, 12ⁱ, 12ⁱⁱ, 12ⁱⁱⁱ, 12^{iv}, 12^{v}, 12^{vi}. For example, in one set of embodiments, a lithium-ion rechargeable battery 54, when fully charged, has a sufficient capacity to power the second aspect control unit 30, 30.2 for several days.

In one set of embodiments, the second aspect control unit 30, 30.2 cooperates with six auscultation sensors 12, 12ⁱ, 12ⁱⁱ, 12ⁱⁱⁱ, 12^{iv}, 12^{v}, 12^{vi}, four of which have a relatively-lower frequency range for sensing heart sounds, with a −24 dB sensitivity and an 80 dB Signal-to-Noise ratio, having a 9.7 mm diameter and a 5 mm height; and two of which have a relatively higher frequency range with a −27 dB sensitivity and a 77 dB Signal-to-Noise ratio, having an 8 mm diameter and a 3 mm height, wherein each of the auscultation sensors 12, 12ⁱ, 12ⁱⁱ, 12ⁱⁱⁱ, 12^{iv}, 12^{v}, 12^{vi} incorporates a microphone that is powered with a low-noise bias voltage supplied by the associated sensor wire-cable 24. For each auscultation sensor 12, 12ⁱ, 12ⁱⁱ, 12ⁱⁱⁱ, 12^{iv}, 12^{v}, 12^{vi}, the associated auscultation signal 37 is first filtered and amplified by a high-pass filter 200, and then further amplified and filtered with an anti-aliasing low-pass filter 202, so as to generate a resulting signal-conditioned auscultation signals 37′. In one set of embodiments, the high-pass filter 200 has a cutoff frequency of 12 Hz for the relatively-low frequency auscultation sensors 12, 12ⁱ, 12ⁱⁱ, 12ⁱⁱⁱ, 12^{iv}, and a cutoff frequency of 56 Hz for the relatively-low frequency auscultation sensors 12, 12^{v}, 12^{vi}; and the anti-aliasing low-pass filter 202 has a cutoff frequency of 1.7 KHz for each of the auscultation sensors 12, 12ⁱ, 12ⁱⁱ, 12ⁱⁱⁱ, 12^{iv}, 12^{v}, 12^{vi}.

The signal-conditioned auscultation signals 37′ from the anti-aliasing low-pass filter 202 is converted from analog to digital form by an analog-to-digital converter (ADC) 204, which, in one set of embodiments, provides for 24-bit simultaneous sampling of eight channels at a 4 KHz sampling rate, and for which the associated internal registers are accessible by the Micro-Controller Unit (MCU) 184 via the SPI bus, and from which the digitized data is transferred to the Micro-Controller Unit (MCU) 184 via the SSP1 bus thereof operating as a Time-Division Multiplexing (TDM) bus, with buffering therebetween to reduce or minimize noise.

The second aspect control unit 30, 30.2 further incorporates a digital-to-analog converter (DAC) 206, which, in one set of embodiments, provides for conversion of 24-bit data of the digitized signal-conditioned auscultation signal 37′—from a selected auscultation sensor 12, 12ⁱ, 12ⁱⁱ, 12ⁱⁱⁱ, 12^{iv}, 12^{v}, 12^{vi}—that is received from the Micro-Controller Unit (MCU) 184 over the I2S bus thereof, for example, at the same sampling rate (e.g. 4 KHz) as the analog-to-digital converter (ADC) 204, with buffering therebetween to reduce or minimize noise. For example, in one set of embodiments, the digital-to-analog converter (DAC) 206 incorporates a built-in voltage reference and a built-in analog output filter, and also provides for interpolation. In one set of embodiments, although the digital-to-analog converter (DAC) 206 provides for generating a stereo audio signal, only the left channel is used for audio output. The output of the digital-to-analog converter (DAC) 206 is filtered with an RC low-pass filter (LPF) 208, amplified by a class-D controllable-gain audio amplifier 210, and then output to one or more electro-static-discharge-protected sockets 44 for communication to a listening device 43 for use by a health care practitioner HCP. The gain of the controllable-gain audio amplifier 210 is controlled by the Micro-Controller Unit (MCU) 184 via the I2C bus responsive to the volume-adjustment touch-switches 52 of the membrane-switch-based user-interface control panel 190, wherein the output of the controllable-gain audio amplifier 210 is further filtered by an RC low-pass filter to reduce switching noise.

In one set of embodiments, the Micro-Controller Unit (MCU) 184 can be debugged and programmed via a Joint Test Action Group (JTAG) bus, and the UARTO bus of the Micro-Controller Unit (MCU) 184 is reserved for bootloader and test purposes.

In one set of embodiments, the Micro-Controller Unit (MCU) 184 is in communication—via the SSPO bus thereof—with a WiFi interface 138 that provides for communication with a remote computing platform 128 via an associated WiFi antenna 152, for example, so as to provide for transmitting signal-conditioned auscultation signals 37′ requested by the remote computing platform 128, or for off-loading data from the second aspect control unit 30, 30.2 to the remote computing platform 128 for storage or further processing.

It should be understood that the number of auscultation sensors 12 that can be used on a given patient 16 is not limiting, nor are the number of auscultation sensors 12 that can be accommodated by aa particular sensor harness-hub 22 or control unit 30. Furthermore, the remote computing platform 128 of the second 10.2 and third 10.3 aspect auscultation systems can be configured to accommodate a plurality of control units 30, 30′, 30″ and associated sensor harness-hubs 22 for use with a single patient 16 so as to provide for expanding the overall channel capacity in support of that patient 16.

Furthermore, the second 10.2 and third 10.3 aspect auscultation systems can be adapted for accessing the associated auscultation signals 134 either primarily or exclusively from a relatively-safe location 130. For example, in accordance with a first alternative aspect, the control unit 30 is configured with sockets 28 by which the plugs 26 of the sensor wire-cables 24 are directly connected, thereby precluding the need for the sensor harness-hub 22 and the associated sensor harness-umbilical-cable 32, with the controls on the control unit 30 only used for initial setup, and with subsequent control being made primarily, if not exclusively, via the remote computing platform 128. In accordance with a second alternative aspect, the control unit 30, 30.1, 30.2 and associated sensor harness-umbilical-cable 32 may be eliminated by incorporating the front-end receiver and low-pass filter LPF, the associated local microcontroller 146, sigma-delta analog-to-digital filter 148 and memory 150, and the WiFi interface 138 of the above-described first aspect control unit 30, 30.1, or the Micro-Controller Unit (MCU) 184, high-pass filter 200, anti-aliasing low-pass filter 202, analog-to-digital converter (ADC) 204, and WiFi interface 138 of the above-described second aspect control unit 30, 30.2, instead in the sensor harness-hub 22, with control thereof being made exclusively via the remote computing platform 128. In accordance with a third alternative aspect, which may be in cooperation with either of the above-described first or second alternative aspects, the remote computing platform 128 may incorporate a Bluetooth® interface to provide for broadcasting auscultation sounds 134′ to a health care practitioner HCP, for example, in the same room 126 as the patient 16, wherein if used within Personal Protective Equipment (PPE), the associated earbuds 43, 42 may not need to be discarded, and might also be used in cooperation with a microphone that would enable the health care practitioner HCP to control by voice the selection and volume of the auscultation sounds 134′ to which they are listening. In accordance with a fourth alternative aspect, which may be in cooperation with either of the above-described first or second alternative aspects, the remote computing platform 128 may be configured to communicate by wire, or wirelessly, with hospital computing platform, the latter of which may provide for wirelessly communicating with any or all of the control unit 30, 30.1, 30.2, a second-alternative-aspect wireless sensor harness-hub 22, or a wireless set of headphones or earbuds 43, 42 worn by the health care practitioner HCP possibly in combination with an above-described wireless microphone, so as to provide for either the remote computing platform 128 or the hospital computing platform to assume primary control of the auscultation process.

The second 10.2 and third 20.3 aspects of the auscultation system 10, 10.2, 10.3 provide for auscultation of patients 16, 16′, 16″ from a remote, relatively-safe location 130 for which the remotely-located health care practitioner HCP′ performing the auscultation need not require personal protective equipment (PPE) that would otherwise be required if personally attending to the patient 16, which thereby both provides for preserving personal protective equipment (PPE) and provides for improving the efficiency of the remotely-located health care practitioner HCP', who does not otherwise have to expend time donning and then removing and disposing the otherwise necessary personal protective equipment (PPE), and also provides for reducing the risk of person-to-person transmission of a contagious disease from the patient 16 to the health care practitioner HCP and then to either or both other patients or other personnel, thereby protecting both health care practitioners HCP and the people and animals with whom they might come in contact after examining an infectious patient 16. The first 10.1, second 10.2 and third 10.3 aspects of the auscultation system 10, 10.1, 10.2, 10.3 provide for health care practitioners HCP to safely listen to auscultation sounds 134′ from a relatively safe distance of at least 2 meters (6 feet) away thereby minimizing the need for close contact therebetween. The use of single-use auscultation sensors 12 and associated sensor wire-cables 24 that can stay on, or with, the patient 16 for an extended period of time provides for reducing the risk of cross-infection-spread of infectious disease from the patient 16 to the health care practitioner HCP, and then from them to others. The auscultation system 10, 10.1, 10.2, 10.3 can be applied to achieve the above benefits in a variety of health-care environments, including, but not limited to hospital emergency rooms, hospital infectious disease isolation rooms, hospital intensive care units, bio-contaminant units, and in radioactive environments. For example, in accordance with one set of embodiments, when used in cooperation with a bio-contaminant unit, the sensor wire-cables 24 are extended through a bio-sealed portal of an associated isopod within which the patent 16 is contained, with the associated sensor harness-hub 22/control unit 30 located in a relative safer region outside the isopod.

In accordance with one set of practices, single-use auscultation sensors 12 are attached to the patient 16 with single-use hydrogel pads 62, 62′, 62″ and used with associated single-use sensor wire-cables 24 to provide for monitoring the patient as frequently as necessary over an extended period of time without requiring direct or close-proximity interaction with an associated PPE-protected health care practitioner HCP, thereby limiting or eliminating the need for PPE protection except when providing other immediate care for the patient 16, for example, when checking for rashes or bedsores, at which time the auscultation sensors 12 might be detached and then reattached to the patient 16 using new single-use hydrogel pads 62, 62′, 62″. For example, in one set of practices, the patient 16 might be checked by a PPE-protected health care practitioner HCP on a daily basis, with the auscultation sensors 12 remaining continuously attached to the patient 16 between such checks, so as to provide for monitoring the auscultation sensors 12 at any time within the intervening periods of time. Then, after the single-use auscultation sensors 12 are finally removed from the patient 16 for example, following a discharge thereof from critical care the single-use auscultation sensors 12 and associated single-use sensor wire-cables 24 are discarded, for example, as medical waste, so as to prevent a spread of infection.

The single usedness of the single-use auscultation sensors 12 is provided for by the associated design thereof that provides for relatively low cost manufacturing, in combination with the use of components that are commercially produced in high volumes to keep recurring cost relatively low. For example, in one set of embodiments, the inverted-bell housing 66 and associated parts 96, 118, or 160 are manufactured using injection-molded plastic (or an injection-molded elastomer for parts 94, or 178 and 180), and the parts are assembled using compression or interference fit, or ultrasonic bonding, without need for glue or an adhesive. Furthermore the single-use auscultation sensor 12 utilizes relatively a microphone 78, 78.1′ that, along with the associated single-use sensor wire-cable 24, is otherwise commercially produced at relatively high volumes for other applications so as to provide for associated relatively-low recurring costs. The single-use auscultation sensors 12 and associated single-use sensor wire-cable 24 do not incorporate any batteries or heavy metals that might otherwise increase associated disposal costs.

While specific embodiments have been described in detail in the foregoing detailed to description and illustrated in the accompanying drawings, those with ordinary skill in the art will appreciate that various modifications and alternatives to those details could be developed in light of the overall teachings of the disclosure. It should be understood, that any reference herein to the term “or” is intended to mean an “inclusive or” or what is also known as a “logical OR”, wherein when used as a logic statement, the expression “A or B” is true if either A or B is true, or if both A and B are true, and when used as a list of elements, the expression “A, B or C” is intended to include all combinations of the elements recited in the expression, for example, any of the elements selected from the group consisting of A, B, C, (A, B), (A, C), (B, C), and (A, B, C); and so on if additional elements are listed. Furthermore, it should also be understood that the indefinite articles “a” or “an”, and the corresponding associated definite articles “the” or “said”, are each intended to mean one or more unless otherwise stated, implied, or physically impossible. Yet further, it should be understood that the expressions “at least one of A and B, etc.”, “at least one of A or B, etc.”, “selected from A and B, etc.” and “selected from A or B, etc.” are each intended to mean either any recited element individually or any combination of two or more elements, for example, any of the elements from the group consisting of “A”, “B”, and “A AND B together”, etc. Yet further, it should be understood that the expressions “one of A and B, etc.” and “one of A or B, etc.” are each intended to mean any of the recited elements individually alone, for example, either A alone or B alone, etc., but not A AND B together. Furthermore, it should also be understood that unless indicated otherwise or unless physically impossible, that the above-described embodiments and aspects can be used in combination with one another and are not mutually exclusive. Accordingly, the particular arrangements disclosed are meant to be illustrative only and not limiting as to the scope of the invention, which is to be given the full breadth of the appended claims, and any and all equivalents thereof.

What is claimed is:

1. A method of auscultation, comprising:
a. adhesively attaching at least one auscultation sensor to a corresponding portion of a skin surface of a patient wherein said at least one auscultation sensor provides for generating a corresponding at least one auscultation signal responsive to a corresponding at least one sound-or-vibration originating from within said patient and in acoustic communication with said at least one auscultation sensor attached to said corresponding portion of said skin surface of said patient; and
b. communicating said corresponding at least one auscultation signal to at least one communications node over at least a corresponding at least one sensor cable in correspondence with said at least one auscultation sensor wherein each said corresponding at least one sensor cable is operatively coupled to a corresponding at least one auscultation sensor said at least one communications node provides for at least one health care practitioner to select and listen at least in real time to said corresponding at least one auscultation signal and said at least one communications node is at a location sufficiently removed from said patient so that said at least one health care practitioner may be at least two meters away from said patient when listening to said corresponding at least one auscultation signal in real time.

2. A method of auscultation as recited in claim 1, wherein the operation of adhesively attaching utilizes an adhesive material that is sufficient to provide for maintaining at least one attachment of said corresponding at least one auscultation sensor to said corresponding portion of said skin surface of said patient for at least 24 hours.

3. A method of auscultation as recited in claim 1, further comprising a sensor hub into which each said corresponding at least one sensor cable is plugged, wherein said sensor hub provides for communicating said corresponding at least one auscultation signal to said at least one communications node.

4. A method of auscultation as recited in claim 3, wherein said sensor hub incorporates a first wireless interface to provide for wirelessly transmitting said corresponding at least one auscultation signal to said at least one communications node.

5. A method of auscultation as recited in claim 3, wherein said sensor hub is operatively coupled via an umbilical cable to a control unit that that can function as said at least one communications node and said umbilical cable provides for communicating each said corresponding at least one auscultation signal from said corresponding at least one auscultation sensor to said control unit.

6. A method of auscultation as recited in claim 1, wherein at least one said at least one communications node comprises a control unit that provides for at least one said at least one health care practitioner to listen to said corresponding at least one auscultation signal in real time, said control unit is operatively coupled to each said corresponding at least one auscultation sensor said control unit provides for selecting and indicating which said corresponding at least one auscultation sensor is to be listened to in real time, and said control unit provides for controlling an audio signal level of said corresponding at least one auscultation signal from said corresponding at least one auscultation sensor being listened to in real time.

7. A method of auscultation as recited in claim 6, wherein said control unit is operatively coupled to said corresponding at least one auscultation sensor via an umbilical cable between said control unit and a sensor hub (into which each said corresponding at least one sensor cable is plugged so as to provide for communicating said corresponding at least one auscultation signal to said at least one communications node.

8. A method of auscultation as recited in claim 6, wherein said control unit incorporates at least one socket for operative connection to at least one listening device so as to provide for said at least one health care practitioner to listen to said corresponding at least one auscultation signal in real time.

9. A method of auscultation as recited in claim 8, wherein said at least one listening device is a listening device selected from the group consisting of at least one headphone and at least one earbud.

10. A method of auscultation as recited in claim 6, wherein said control unit is powered by a battery further comprising indicating a state-of-charge of said battery.

11. A method of auscultation as recited in claim 10, wherein said battery is external of said control unit.

12. A method of auscultation as recited in claim 6, wherein said control unit incorporates a first wireless interface to provide for wirelessly transmitting said corresponding at least one auscultation signal to at least one other said at least one communications node.

13. A method of auscultation as recited in claim 1, wherein at least one said at least one communications node comprises a remote device that provides for at least one said at least one health care practitioner to listen to said corresponding at least one auscultation signal from said corresponding at least one auscultation sensor in real time without needing to utilize personal protective equipment to avoid becoming infected by a contagiously-infected said patient said remote device is in communication with said corresponding at least one auscultation sensor said remote device provides for selecting which at least one said corresponding at least one auscultation sensor is to be listened to in real time, and said remote device provides for controlling an audio signal level of said corresponding at least one auscultation signal from said corresponding at least one auscultation sensor being listened to in real time.

14. A method of auscultation as recited in claim 4, wherein at least one said at least one communications node comprises a remote device that provides for at least one said at least one health care practitioner to listen to said corresponding at least one auscultation signal from said corresponding at least one auscultation sensor in real time without needing to utilize personal protective equipment to avoid becoming infected by a contagiously-infected said patient said remote device is in communication with said corresponding at least one auscultation sensor said remote device provides for selecting which at least one said corresponding at least one auscultation sensor is to be listened to in real time, said remote device provides for controlling an audio signal level of said corresponding at least one auscultation signal from said corresponding at least one auscultation sensor being listened to in real time, and said remote device incorporates a second wireless interface to provide for wirelessly receiving said corresponding at least one auscultation signal from said sensor hub.

15. A method of auscultation as recited in claim 12, wherein said at least one other said at least one communications node comprises a remote device that provides for at least one said at least one health care practitioner to listen to said corresponding at least one auscultation signal from said corresponding at least one auscultation sensor in real time without needing to utilize personal protective equipment to avoid becoming infected by a contagiously-infected said patient said remote device is in communication with to said corresponding at least one auscultation sensor said remote device provides for selecting which at least one said corresponding at least one auscultation sensor is to be listened to in real time, said remote device provides for controlling said audio signal level of said corresponding at least one auscultation signal from said corresponding at least one auscultation sensor being listened to in real time, and said remote device incorporates a second wireless interface to provide for wirelessly receiving said corresponding at least one auscultation signal from said control unit.

16. A method of auscultation as recited in claim 13, wherein said remote device incorporates at least one socket for operative connection to at least one listening device so as to provide for said at least one health care practitioner to listen to said corresponding at least one auscultation signal in real time.

17. A method of auscultation as recited in claim 13, wherein said remote device incorporates a plurality of sockets for operative connection to a corresponding plurality of listening devices so as to provide for each of a plurality of health care practitioners to listen in real time to a corresponding auscultation signal selected from said corresponding at least one auscultation signal.

18. A method of auscultation as recited in claim 16, wherein said at least one listening device is a listening device selected from the group consisting of at least one headphone and at least one earbud.

19. A method of auscultation as recited in claim 1, wherein at least one said corresponding at least one auscultation sensor comprises:
a. an inverted-bell housing comprising an internal surface that bounds an open-ended cavity, and an annular rim surrounding an open end of said open-ended cavity, wherein said annular rim provides for the operation of adhesively attaching said at least one said corresponding at least one auscultation sensor to said skin surface of said patient in cooperation with an adhesive material disposed between said annular rim and said skin surface of said patient; and
b. an acoustic port though said inverted-bell housing, wherein said acoustic port in in acoustic communication with said open-ended cavity; and
c. an acoustic transducer, wherein said acoustic transducer is in acoustic communication with said acoustic port so as to provide for receiving a sound from within said open-ended cavity, and said acoustic transducer provides for generating an electrical auscultation signal responsive to said sound from within said open-ended cavity; wherein said corresponding at least one sensor cable comprises an electrical cable operatively coupled to said acoustic transducer, said electrical cable is terminated with a first portion of a connector pair that provides for mating with a corresponding second portion of said connector pair, so as to provide for communicating said electrical auscultation signal, or a signal responsive thereto, as said corresponding at least one auscultation signal from said acoustic transducer to said at least one communications node via said sensor cable connector pair.

20. A method of auscultation as recited in claim 19, wherein a mouth of said inverted-bell housing bounded by said annular rim incorporates a grate to provide for resisting an intrusion of said skin surface of said patient into said open-ended cavity.

21. A method of auscultation as recited in claim 19, wherein said acoustic port is located at or proximate to an apex of said open-ended cavity.

22. A method of auscultation as recited in claim 19, wherein said acoustic transducer comprises a microphone.

23. A method of auscultation as recited in claim 19, wherein said acoustic transducer comprises a MEMS acoustic transducer.

24. A method of auscultation as recited in claim 19, wherein said acoustic transducer is at least partially acoustically insulated from background noise by an elastomeric material at least partially surrounding said acoustic transducer.

25. A method of auscultation as recited in claim 1, wherein at least one said corresponding at least one auscultation sensor incorporates a sound-deadening material to provide for attenuating a reception of background acoustic noise by said at least one said corresponding at least one auscultation sensor.

26. A method of auscultation, comprising:
a. adhesively attaching at least one auscultation sensor to a corresponding portion of a skin surface of a patient wherein said at least one auscultation sensor provides for generating a corresponding at least one auscultation signal over a corresponding associated at least one sensor cable responsive to a corresponding at least one sound-or-vibration originating from within said patient and in acoustic communication with said at least one auscultation sensor;
b. listening to or processing at least one said at least one auscultation signal from at least one location at least two meters away from said patient wherein at least one said at least one location is cable-connected to said at least one auscultation sensor by at least said corresponding associated at least one sensor cable;
c. maintaining an attachment of said at least one auscultation sensor to said patient for a duration of attachment of at least 24 hours;
d. removing said at least one auscultation sensor from said patient following said duration of attachment; and
e. discarding said at least one auscultation sensor and said corresponding associated at least one sensor cable following the removal of said at least one auscultation sensor from said patient.
